SEQUENCE LISTING (1 sequence)

SEQ ID NO: 1 - DNA, 3747 bp, Homo sapiens
gaattcggccgctgagagaccctggacactgttcctgctccctcgccaccaaaacttctcctccagtcccctcccctgcaggaccatcgcccgcagcctctgcacctgttttcttgtgtttaagggtggggtttgcccccctccccacgctcccatctctgatcctcccaccttcacccgcccaccccgcgagtgagtgcggtgcccaggcgcgcttggcctgagaggtcggcagcagacccggcagcgccaaccgcccagccgctctgactgctccggctgcccgcccgcgcggcgcgggctgtcctggaccctaggaggggacggaaccggacttgcctttgggcaccttccagggccctctccaggtcggctggctaatcatcggacagacggactgcacacatcttgtttccgcgtctccgcaaaaacgcgaggtccaggtcagttgtagactcttgtgctggttgcaggccaagtggacctgtactgaaaatgggtccaataggtgcagaggctgatgagaaccagacagtggaagaaatgaaggtggaacaatacgggccacaaacaactcctagaggtgaactggtccctgaccctgagccagagcttatagatagtaccaagctgattgaggtacaagttgttctcatattggcctactgctccatcatcttgcttggggtaattggcaactccttggtgatccatgtggtgatcaaattcaagagcatgcgcacagtaaccaactttttcattgccaatctggctgtggcagatcttttggtgaacactctgtgtctaccgttcactcttacctataccttaatgggggagtggaaaatgggtcctgtcctgtgccacctggtgccctatgcccagggcctggcagtacaagtatccacaatcaccttgacagtaattgccctggaccggcacaggtgcatcgtctaccacctagagagcaagatctccaagcgaatcagcttcctgattattggcttggcctggggcatcagtgccctgctggcaagtcccctggccatcttccgggagtattcgctgattgagatcattccggactttgagattgtggcctgtactgaaaagtggcctggcgaggagaagagcatctatggcactgtctatagtctttcttccttgttgatcttgtatgttttgcctctgggcattatatcattttcctacactcgcatttggagtaaattgaagaaccatgtcagtcctggagctgcaaatgaccactaccatcagcgaaggcaaaaaaccaccaaaatgctggtgtgtgtggtggtggtgtttgcggtcagctggctgcctctccatgccttccagcttgccgttgacattgacagccaggtcctggacctgaaggagtacaaactcatcttcacagtgttccacattatcgccatgtgctccacttttgccaatccccttctctatggctggatgaacagcaactacagaaaggctttcctctcggccttccgctgtgagcagcggttggatgccattcactctgaggtgtccgtgacattcaaggctaaaaagaacctggaggtcagaaagaacagtggccccaatgactctttcacagaggctaccaatgtctaaggaagctgtggtgtgaaaatgtatggatgaattctgaccagagctatgaatctggttgatggcggctcacaagtgaaaactgatttcccattttaaagaagaagtggatctaaatggaagcatctgctgtttaattcctggaaaactggctgggcagagcctgtgtgaaaatactggaattcaaagataaggcaacaaaatggtttacttaacagttggttgggtagtaggttgcattatgagtaaaagcagagagaagtacttttgattattttcctggagtgaagaaaacttgaacaagaaattggtattatcaaagcattgctgagagacggtgggaaaataagttgactttcaaatcacgttaggacctggattgaggaggtgtgcagttcgctgctccctgcttggcttatgaaaacaccactgaacagaaatttctccagggagccacaggctctccttcatcgcattttgatttttttgttcattctctagacaaaatccatcagggaatgctgcaggaaacgattgccaactatacgaatggcttcgaggagataaactgaaatttgctatataattaatattttggcagatgataggggaactcctcaacactcagtgggccaattgttcttaaaaccaattgcacgtttggtgaaagtttcttcaactctgaatcaaaagctgaaattctcagaattacaggaaatgcaaaccatcatttaatttctaatttcaagttacatccgctttatggagatactatttagataacaagaatacaacttgatacttttattgttatacctttttgaacatgtatgatttctgttgttattcctattggagctaagtttgtctacactaaaatttaaatcagactagagaataatttttgtggcatgttgtaacatttcacagtatttacaagctatttttgcacaggtacatagctctcatgtatttaaagaacactgcagtgttattttctttgaaattcatcctccacggacccattcatactaaataaaacaatgtaattacattaaaatggacctatctgtaagaggtactaaaaacactggattcatttcatcttgcaaatgttgtatttcaaaccagtttcacataagttatttgtcttcttttcaaaataattagctatatttttatataatatgaatatatacataaaaattgtttctataaattgtagaacatagatgctacagtattttttatttaattatattatgaataaaattgttatttcaatagtacccaaccaaagatgcttaaaaaccttctatgttcataaaaaataacaactgagatgttaaaatagtcatacgtctttagatgctattaaagtttcattagtcatatttttgtaaatatgacagaatttgtgaatatatttttaaagcaaaaaacttcaacatgcatatgatatatagttacaacattaattttatgaactggagagctttactttgtggatatatttaaaattcatattatagctcctattaaattccttccatgatagatataaaggactggtttttaagtgcactgcacttctggaatactgaaaaagaatgaaaacaatatgttagattaggtgtaagactttaagaagcgaacaaaaagtaatgtatatctgtaatatataatcaaatgattcatttttctgttagactaggcaaattgttcaaaaataacctttttgtcttttaagtagcagtcactttgcttaagatgctaatagaaaactgtggttaaagatttaccctccctcttggtgaattattacactgtaagaaatgtatatgctactgtgttacatgttgtattagtaaattattagaatccaattaatgattcaattaacatatatcttatccaattcattatgtcaattcattaataaaataccttttatgtagaggctttatgttgcaattaaaaagttgggaaaatgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2013/0261011 A1
(43) Pub. Date: Oct. 3, 2013

(54) ANALYZING NEONATAL SALIVA AND READINESS TO FEED

(71) Applicant: TUFTS MEDICAL CENTER, INC., BOSTON, MA (US)

(72) Inventors: JILL L. MARON, SHARON, MA (US); KIRBY L. JOHNSON, NORTH ATTLEBORO, MA (US); DIANA W. BIANCHI, CHARLESTOWN, MA (US)

(73) Assignee: TUFTS MEDICAL CENTER, INC., BOSTON, MA (US)

(21) Appl. No.: 13/796,843

(22) Filed: Mar. 12, 2013

Related U.S. Application Data
(60) Provisional application No. 61/618,184, filed on Mar. 30, 2012.

(51) Int. Cl. C12Q 1/68

(52) U.S. Cl. CPC C12Q 1/6809
USPC 506/9; 435/6.11

(57) ABSTRACT

The present invention provides systems for assessing neonatal development and/or conditions by analyzing neonatal saliva RNA. Methods of identifying genes involved in neonatal development and/or conditions affecting neonates, are provided. Methods of determining a diagnosis of a neonate comprising detection of one or more differentially expressed genes are also provided.

### CROSS-REFERENCE TO RELATED APPLICATIONS

This application claims the benefit under 35 U.S.C. §119(e) of U.S. Provisional Application No. 61/618,184, filed Mar. 30, 2012, the contents of which are incorporated herein by reference in their entirety.

### GOVERNMENT SUPPORT

The invention was made with government support under Grant No. K08 HD059819-03 awarded by the National Institute of Child Health & Human Development. The government has certain rights in the invention.

### SEQUENCE LISTING

The present specification makes reference to a Sequence Listing (submitted electronically as a .txt file named “Sequence Listing.txt on Mar. 12, 2013). The .txt file was generated on Mar. 8, 2013 and is 5.18 kb in size. The entire contents of the Sequence Listing are herein incorporated by reference.

### BACKGROUND

Complications associated with feeding can cause significant infant morbidities, and in particular affect the majority of neonates in neonatal intensive care units (NICU). These complications can include gastroesophageal reflux (GERD), feeding intolerance, uncoordinated and immature feeding patterns, and/or inflammatory and necrotic processes of the bowel such as necrotizing enterocolitis (NEC). Such complications often lead to prolonged hospitalizations, medication administration, parental anxiety, and significant neonatal morbidity and mortality.

Development of diagnostics and therapies for such complications has been hindered by the fragility of premature neonates, which excludes them from studies involving invasive procedures. Their limited blood volumes make it impractical or impossible to draw blood from them frequently.

### SUMMARY

The present invention encompasses the recognition that accurate assessment of readiness to feed, particularly in premature infants, could significantly reduce infant morbidities, or risks of such morbidities. Current standard of care in newborn medicine is to have caregivers subjectively assess readiness of an infant to feed by mouth. However, in accordance with the present disclosure, the inventors note that when such an assessment is inaccurate, resulting morbidities can be significant, and can cause long-term health consequences, particularly for premature babies.

Among other things, the present disclosure provides systems for assessing infant readiness to feed, including premature infant readiness to feed. In some embodiments, provided systems are noninvasive. In some embodiments, provided systems include analysis of saliva samples.

In some embodiments, provided systems involve transcriptomic analysis of saliva (e.g., from neonates). In some embodiments, provided systems involve analysis of nucleic acids in saliva. In some embodiments, provided systems involve quantification of one or more particular markers in saliva. In some embodiments, provided systems involve detection and/or quantification of levels and/or activity of neuropeptide Y2 receptor (NPY2R).

The present disclosure provides the particular surprising finding that effective analysis, particularly including quantification of one or more markers, and/or particularly including genetic analysis, can be performed on small volumes (e.g. volumes less than about 50 μL, 45 μL, 40 μL, 35 μL, 30 μL, 25 μL, 20 μL, 10 μL, 9 μL, 8 μL, 7 μL, 6 μL, 5 μL, 4 μL, 3 μL, 2 μL, or even 1 μL or less) of saliva.

### BRIEF DESCRIPTION OF THE DRAWING

FIG. 1 depicts a representative plot from a BioAnalyzer analysis of amplified total RNA from neonatal saliva sample. Such plots are typically used to evaluate quantity and quality of nucleic acids such as RNA. Time in seconds is plotted on the x-axis and fluorescence is plotted on the y-axis. The area under the curve represents concentration of total RNA extracted from saliva sample. In the BioAnalyzer result depicted, the concentration of amplified total RNA was about 849 ng/μL.

FIG. 2 outlines time points for salivary collection for experiments described in Examples 3-4.

FIG. 3: shows NPY2R gene expression and advancing post-conceptual age in weeks for all infants.

FIG. 4: depicts NPY2R gene expression and feeding status in term infants.

FIG. 5: illustrates NPY2R gene expression and feeding status for all infants.

### DEFINITIONS

Throughout the specification, several terms are employed that are defined in the following paragraphs.

As used herein, the terms “about” and “approximately,” in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the term “biomarker” has its meaning as understood in the art. In some embodiments, the term refers to an indicator that provides information about, among other things, a process, condition, developmental stage, or outcome of interest, e.g., a neonate's developmental readiness for feeding. In many embodiments, the value (e.g., the level at which it is present or the level at which it is active) of such an indicator is correlated with a process, condition, developmental stage, or outcome of interest. In some embodiments, the term “biomarker” refers to a molecule that is the subject of an assay or measurement, the result of which provides information about (e.g. correlates with) a process, condition, developmental stage, or outcome of interest. For example, an elevated expression or activity level of a particular gene can be an indicator that a subject has a particular condition. The expression and/or activity level of the gene or gene product, an elevated expression or activity level (e.g., above a particular threshold) of the gene, and/or the gene expression product itself, can each be referred to as “biomarkers.”

As used herein, the term “complementary” refers to nucleic acid sequences that base-pair according to the standard Watson-Crick complementary rules, or that are capable of hybridizing to a particular nucleic acid segment under relatively stringent conditions. Nucleic acid polymers are optionally complementary across only portions of their entire sequences.

As used herein, the term “differentially expressed” in reference to genes refers to the state of having a different expression pattern or level depending on the type of cell, tissue, and/or sample, from which the gene expression products are derived. “Differentially expressed” genes may be upregulated or downregulated in the cell, tissue, and/or samples as compared to controls. For example, a gene that is downregulated in samples from a subject that has undergone a developmental transition (such as the ability to swallow) as compared to a subject who has not can also be said to be “differentially expressed.”

As used herein, the term “enteral feeding” refers to delivery of liquid feeding to the gastrointestinal tract via a tube.

As used herein, the phrase “feeding capability” refers collectively to an individual's readiness to feed and feeding tolerance.

As used herein, the phrase “feeding intolerance” refers the inability of an individual (e.g., a neonate) to achieve and/or maintain full enteric feeds. Likewise “feeding tolerance” as used herein refers to the ability of an individual (e.g., a neonate) to achieve and/or maintain full enteric feeds.

As used herein, terms “fluorophore”, “fluorescent moiety”, “fluorescent label”, “fluorescent dye”, and “fluorescent labeling moiety” are used interchangeably. They refer to a molecule that, in solution and upon excitation with light of appropriate wavelengths, emits light back. Numerous fluorescent dyes of a wide variety of structures and characteristics are suitable for use in the practice of this invention. Similarly, methods and materials are known for fluorescently labeling nucleic acids (see, for example, Haugland (1994)). In choosing a fluorophore, it is preferred that the fluorescent molecule absorbs light and emits fluorescence with high efficiency (i.e., high molar absorption coefficient and fluorescence quantum yield, respectively) and is photostable (i.e., it does not undergo significant degradation upon light excitation within the time necessary to perform the analysis).

As used herein, the term “full gastric feeds” refers to any infant receiving all feeds via a nasogastric or oral gastric tube.

As used herein, the term “full oral feeds” refers to any infant receiving all feeds orally, without any nutritional supplementation provided through a nasogastric or oral gastric tube.

As used herein, the term “gene” refers to a discrete nucleic acid responsible for a discrete cellular product and/or performing one or more intracellular or extracellular functions. In some embodiments, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. Such gene and regulatory sequences may be derived from the same natural source, or may be heterologous to one another. In some embodiments, a gene does not encode proteins but rather provide templates for transcription of functional RNA molecules such as tRNAs, rRNAs, etc. Alternatively or additionally, in some embodiments, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA), or the product of subsequent downstream processing events (e.g., splicing, RNA processing, translation). In some embodiments, a gene product is a protein produced by translation of an mRNA. In some embodiments, gene products are RNAs that are modified by processes such as capping, polyadenylation, methylation, and editing, proteins post-translationally modified, and proteins modified by, for example, methylation, acetylation, phosphorylation, ubiquitination, ADP-ribosylation, myristilation, and glycosylation.

As used herein, the term “gene expression array” refers to an array comprising a plurality of genetic probes immobilized on a substrate surface that can be used for quantitation of mRNA expression levels. In the context of the present invention, the term “array-based gene expression analysis” is used to refer to methods of gene expression analysis that use gene-expression arrays. The term “genetic probe”, as used herein, refers to a nucleic acid molecule of known sequence, which has its origin in a defined region of the genome and can be a short DNA sequence (or oligonucleotide), a PCR product, or mRNA isolate. Genetic probes are gene-specific DNA sequences to which nucleic acids from a test sample of saliva RNA are hybridized. Genetic probes specifically bind (or specifically hybridize) to nucleic acid of complementary or substantially complementary sequence through one or more types of chemical bonds, usually through hydrogen bond formation.

As used herein, the term “gestational age” refers to age of an embryo, fetus, or neonate as calculated from the first day of the mother's last menstrual period. In humans, the gestational age may count the period of time from about two weeks before fertilization takes place. Gestational age is most accurately used up to and including the day of birth (e.g., infant was born at a gestational age of 25 4/7 weeks). From the first day of life onwards, the neonate's age should be referred to as post-conceptual age.

As used herein, the term “isolated” when applied to RNA means a molecule of RNA or a portion thereof, which (1) by virtue of its origin or manipulation, is separated from at least some of the components with which it was previously associated; or (2) was produced or synthesized by the hand of man.

As used herein, the terms “labeled”, “labeled with a detectable agent” and “labeled with a detectable moiety” are used interchangeably. They are used to specify that a nucleic acid molecule or individual nucleic acid segments from a sample can be visualized, for example, following binding (i.e., hybridization) to genetic probes. In hybridization methods, samples of nucleic acid segments may be detectably labeled before the hybridization reaction or a detectable label may be selected that binds to the hybridization product. Preferably, the detectable agent or moiety is selected such that it generates a signal which can be measured and whose intensity is related to the amount of hybridized nucleic acids. In array-based methods, the detectable agent or moiety is also preferably selected such that it generates a localized signal, thereby allowing spatial resolution of the signal from each spot on the array. Methods for labeling nucleic acid molecules are well known in the art (see below for a more detailed description of such methods). Labeled nucleic acid fragments can be prepared by incorporation of or conjugation to a label, that is directly or indirectly detectable by spectroscopic, photochemical, biochemical, immunochemical, electrical, optical, or chemical means. Suitable detectable agents include, but are not limited to: various ligands, radionuclides, fluorescent dyes, chemiluminescent agents, microparticles, enzymes, colorimetric labels, magnetic labels, and haptens. Detectable moieties can also be biological molecules such as molecular beacons and aptamer beacons.

As used herein, the term “messenger RNA” or “mRNA” refers a form of RNA that serves as a template for protein biosynthesis. In many embodiments, the amount of a particular mRNA (i.e., having a particular sequence, and originating from a particular same gene) reflects the extent to which the gene encoding the mRNA has been “expressed.”

As used herein, the terms “microarray,” “array” and “biochip” are used interchangeably and refer to an arrangement, on a substrate surface, of multiple nucleic acid molecules of known sequences. Each nucleic acid molecule is immobilized to a “discrete spot” (i.e., a defined location or assigned position) on the substrate surface. The term “microarray” more specifically refers to an array that is miniaturized so as to require microscopic examination for visual evaluation. Arrays used in the methods of the invention are preferably microarrays.

As used herein, the terms “neonate,” and “newborn” are used interchangeably and refer to subjects who have recently been born. In some embodiments, the neonate is a human within the first three months of being born. In some embodiments, the neonate is a human within the first two months of being born. In some embodiments, the neonate is a human within the first month of being born. In some embodiments of the invention, the neonate is prematurely born; in some such embodiments, the premature neonate is a human neonate born between 23 and 37 weeks' gestational age.

As used herein, the term “NPY2R” refers to a gene encoding a neuropeptide Y2 receptor polypeptide, or a nucleic acid having substantial identity to a gene encoding an a neuropeptide Y2 receptor polypeptide. The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

As used herein, the terms “nucleic acid” and “nucleic acid molecule” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products.

As used herein, the term “oligonucleotide” refers to usually short strings of DNA or RNA to be used as hybridizing probes or nucleic acid molecule array elements. These short stretches of sequence are often chemically synthesized. The size of the oligonucleotide depends on the function or use of the oligonucleotides. When used in microarrays for hybridization, oligonucleotides can comprise natural nucleic acid molecules or synthesized nucleic acid molecules and comprise between 5 and 150 nucleotides, preferably between about 15 and about 100 nucleotides, more preferably between 15 and 30 nucleotides and most preferably, between 18 and 25 nucleotides complementary to mRNA.

As used herein, the term “oral feeding” refers to the delivery of feeding to the mouth.

As used herein, the term “partial gastric feeds” refers to any neonate who is receiving at least some of his/her feeds via a nasogastric or oral gastric tube. In some embodiments, the infant may receive additional nutritional supplementation via an intravenous line. In some embodiments, the infant may receive additional nutritional supplementation via oral feeding.

As used herein, the term “partial oral feeds” refers to any neonate who can take some (range: 1-99%) but not all of his/her feeds orally. In some embodiments, the infant may receive additional nutritional supplementation via an intravenous line. In some embodiments, the infant may receive additional nutritional supplementation via gastric tube feeding.

As used herein, the term “post-conceptual age” refers to age of a neonate as the time elapsed since conception or the time elapsed from the first day of the mother's last menstrual period. The term “post-conceptual age” will be used to date all infants from their day of birth onwards. The term “gestational age” will be used to date all fetuses/infants up to their day of birth.

As used herein, the terms “premature neonate” and “preterm neonate” are used interchangeably and refer to neonates who are born before the full term of a typical pregnancy. In some embodiments, the premature neonate is a human born before 37 weeks' gestation.

As used herein, the term “RNA transcript” refers to the product resulting from transcription of a DNA sequence. When the RNA transcript is the original, unmodified product of a RNA polymerase catalyzed transcription, it is referred to as the primary transcript. An RNA transcript that has been processed (e.g., spliced, etc.) will differ in sequence from the primary transcript; a fully processed transcript is referred to as a “mature” RNA. The term “transcription” refers to the process of copying a DNA sequence of a gene into an RNA product, generally conducted by a DNA-directed RNA polymerase using the DNA as a template. A processed RNA transcript that is translated into protein is often called a messenger RNA (mRNA).

As used herein, the phrase “readiness to feed” refers to a subject's ability to transition from enteral feeding to oral feeding. “Readiness to feed” may be indicative of developmental progress and/or improvement with respect to a medical condition.

As used herein, the term “saliva” refers to a biological fluid produced in and secreted from salivary glands and found in the mouths of humans and other animals. Saliva is comprised of water, digestive enzymes, proteins, hormones, electrolytes, mucus, antibacterial compounds, and nucleic acids DNA and RNA, and is a component of the digestion system. In some embodiments, a saliva sample is obtained by suction from the oropharynx.

As used herein, the term “statistically significant number” refers to a number of samples (analyzed or to be analyzed) that is large enough to provide reliable data.

As used herein, the terms “subject” and “individual” are used herein interchangeably. They refer to a human or another animal (e.g., mouse, rat, rabbit, dog, cat, cattle, swine, sheep, horse, or primate) that can be afflicted with or is susceptible to a disease, disorder, condition, or complication (e.g., necrotizing enterocolitis) but may or may not have the disease or disorder. In many embodiments, the subject is a human being. In many embodiments, the subject is a neonate. In some embodiments, the subject is a premature neonate.

As used herein, the term “susceptible” means having an increased risk for and/or a propensity for something, i.e., a condition such as necrotizing enterocolitis. The term takes into account that an individual “susceptible” for a condition may never be diagnosed with the condition.

As used herein, the terms “mature neonate” and “term neonate” are used interchangeably and refer to neonates who are either born after the full term of a typical pregnancy or have a post-conceptual age of ≧37 weeks. In some embodiments, the term neonate is a human born at or after 37 weeks' gestation.

### DETAILED DESCRIPTION OF CERTAIN EMBODIMENTS

As mentioned above, the present invention provides technologies for detecting and/or identifying genes that are involved in neonatal development and/or in conditions affecting neonates. The present invention also provides technologies for diagnosing a neonate.

The present inventors have recognized, among other things, that analyzing neonatal salivary RNA may provide valuable information about neonatal development and/or disease. Although some success has been reported in obtaining and analyzing salivary RNA from adults, to the knowledge of the present inventors, no attempts have heretofore been made to obtain and analyze salivary RNA from neonates. This lack of attempt by others may reflect, among other things, an expectation of failure due to certain difficulties in obtaining and analyzing RNA from neonates. For example, whereas sufficient quantities of saliva for RNA extraction may easily be obtained from adults, much smaller quantities can be obtained from neonates, thus limiting the amount of starting material from which RNA can be obtained. Limited amounts of starting material present challenges for certain analyses, especially those involving large quantities of RNA such as genome-wide gene expression analyses. Such challenges may be exacerbated in premature neonates and/or neonates suffering from a disease or condition, who are often even smaller in size and are often supported by feeding tubes and/or other life support paraphernalia.

The present inventors have overcome these challenges and successfully demonstrated extraction, amplification, and analyses of neonatal salivary RNA. In some embodiments, analyses comprise performing genome-wide (“global”) or other large scale gene expression analyses. To the knowledge of the present inventors, such large scale gene expression analyses have heretofore not been performed on any salivary RNA samples, as reports on adult salivary RNA were limited to analyses of a small subset of genes. Larger scale gene expression analyses on salivary RNA, such as those disclosed herein, may provide insight into many physiological and developmental systems and into relationships between gene products. The present inventors have also recognized that profiling gene expression (for example, at a global level) of RNA from developing neonates at various points in time provides further insights.

Such insights, as obtained from methods disclosed herein, are especially valuable for understanding developmental processes relevant to neonates, including those neonates with a disease or condition.

I. Neonatal RNA from Saliva

In some embodiments, methods of the invention involve providing neonatal RNA from saliva samples. Saliva samples can be obtained from neonates by, for example, gentle suctioning of the oropharynx. Typically one can obtain between about 100 μL to about 200 μL saliva by gentle suctioning. The present inventors have made significant advancements in the limits of gene detection, and have surprisingly demonstrated successful extraction of RNA and analysis of gene expression analysis from much smaller volumes. The present invention remarkably provides technologies that permit such extraction and analysis from saliva samples having a volume below about 50 μL, 45 μL, 40 μL, 35 μL, 30 μL, 25 μL, 20 μL, or even less. Indeed, in some embodiments, the present invention even provides technologies that permit such extraction and analysis from saliva samples having a volume as small as 10 μL, 8 μL, 7 μL, 4 μL, 3 μL, 2 μL, or even 1 μL or less.

As collecting saliva in accordance with embodiments of the present invention is non-invasive, saliva can be collected repeatedly from the same neonate without harm to the neonate. In some embodiments, saliva is collected serially from the same neonate, and in some such embodiments, saliva is collected at various timepoints in a neonate's development.

In some embodiments, saliva is obtained from premature neonates. In some embodiments, saliva is collected from premature neonates that are underdeveloped and/or underweight. Such neonates often have problems relating to feeding, breathing, and/or staying warm. For example, saliva may be collected from human premature neonates that were born at 37 weeks' gestation. In some embodiments, saliva is collected from human premature neonates born at 32 weeks' gestation. In some embodiments, saliva is collected from human premature neonates born at 24 weeks' gestation. In some embodiments, saliva is collected from newborns having a post-conceptual age (PCA) within a range having a low end of 26 4/7 to high end of 41 4/7 weeks. In some embodiments, the low end is 23, 24, or 25 weeks; in some embodiments the high end is 43, 45, 47, 48, 49, or 52 weeks.

### Isolation of Neonatal Saliva RNA

Neonatal RNA for use in the methods of the present invention is typically isolated from a sample of saliva obtained from a neonate. Such isolation may be carried out by any suitable method of RNA isolation or extraction.

In certain embodiments, neonatal RNA is obtained by treating a sample of saliva such that neonatal RNA present in the sample of saliva is extracted. In some embodiments, neonatal salivary RNA is extracted from a sample of saliva containing cells and/or cellular material.

Neonatal RNA may also be obtained by isolating cells from the sample of saliva, optionally cultivating these isolated cells, and extracting RNA from the cells. In such cases, neonatal salivary RNA consists essentially of neonatal RNA from the cultured cells.

Neonatal RNA may also be obtained from the salivary supernatant. In such cases, supernatant is isolated from the cells following centrifugation. In such cases, neonatal salivary RNA consists exclusively of cell-free RNA.

In some embodiments, before isolation or extraction of neonatal RNA, the sample of saliva material is stored for a certain period of time under suitable storage conditions. In some embodiments, suitable storage conditions comprise temperatures ranging between about 21° C. to about −220° C., inclusive. In some embodiments, samples are stored at about 4° C., at about −10° C., at about −20° C., at about −70° C., or at about −80° C. In some embodiments, samples are stored for less than about 28 days. In some embodiments, samples are stored for more than about twenty-four hours. In some embodiments, before freezing, an RNase inhibitor, which prevents degradation of neonatal RNA by RNases (i.e., ribonucleases), is added to the sample. In some embodiments, the RNase inhibitor is added within two hours of obtaining the sample of salivary material. In some embodiments, the RNAse inhibitor is added within one hour of obtaining the sample of salivary material. In some embodiments, the RNAse inhibitor is added within thirty minutes of obtaining the sample of salivary material. In some embodiments, the RNAse inhibitor is added within ten minutes of obtaining the sample of salivary material. In some embodiments, the RNAse inhibitor is added within five minutes of obtaining the sample of salivary material. In some embodiments, the RNAse inhibitor is added within two minutes of obtaining the sample of salivary material. In some embodiments, the RNase inhibitor is added immediately after obtaining the sample of remaining salivary material. In some embodiments, before RNA extraction, the frozen sample is thawed at 37° C. and mixed with a vortex.

In some embodiments, the sample is frozen (e.g., flash-frozen in liquid nitrogen and dry ice), stored, and thawed; then RNAse inhibitor is added after thawing. In some such embodiments, the RNase inhibitor is added within two hours of thawing. In some embodiments, the RNAse inhibitor is added within one hour of thawing. In some embodiments, the RNAse inhibitor is added within thirty minutes of thawing. In some embodiments, the RNAse inhibitor is added within ten minutes of thawing. In some embodiments, the RNAse inhibitor is added within five minutes of thawing. In some embodiments, the RNAse inhibitor is added within two minutes of thawing.

The most commonly used RNase inhibitor is a natural protein derived from human placenta that specifically (and reversibly) binds RNases (Blackburn et al. (1977), the entire contents of which are herein incorporated by reference). RNase inhibitors are commercially available, for example, from Ambion (Austin, Tex.; as SUPERase•In™), Promega, Inc. (Madison, Wis.; as rRNasin® Ribonuclease Inhibitor) and Applied Biosystems (Framingham, Mass.). In general, precautions for preventing RNases contaminations in RNA samples, which are well known in the art and include the use of gloves, of certified RNase-free reagents and ware, of specifically treated water and of low temperatures, as well as routine decontamination and the like, are used in the practice of the methods of the invention.

Isolating neonatal RNA may include treating the remaining salivary material such that neonatal RNA present in the remaining salivary material is extracted and made available for analysis. Any suitable isolation method that results in extracted saliva neonatal RNA may be used in the practice of the invention. In order to obtain the most accurate assessment of the neonate, it is desirable to minimize artifacts from manipulation processes. Therefore, the number of extraction and modification steps is in some embodiments kept as low as possible.

Methods of RNA extraction are well known in the art (see, for example, Sambrook et al. (1989). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors (Chirgwin et al. (1979)). Isolated total RNA is then further purified from the protein contaminants and concentrated by selective ethanol precipitations, phenol/chloroform extractions followed by isopropanol precipitation (see, for example, Chomczynski and Sacchi (1987)) or cesium chloride, lithium chloride or cesium trifluoroacetate gradient centrifugations (see, for example, Glisin et al (1974) and Stern and Newton (1986)).

In certain methods of the invention, for example those wherein saliva neonatal RNA is subjected to a gene-expression analysis, it may be desirable to isolate mRNA from total RNA in order to allow the detection of even low level messages (Alberts et al. (1994)).

Purification of mRNA from total RNA typically relies on the poly(A) tail present on most mature eukaryotic mRNA species. Several variations of isolation methods have been developed based on the same principle. In a first approach, a solution of total RNA is passed through a column containing oligo(dT) or d(U) attached to a solid cellulose matrix in the presence of high concentrations of salts to allow the annealing of the poly(A) tail to the oligo(dT) or d(U). The column is then washed with a lower salt buffer to remove and release the poly(A) mRNAs. In a second approach, a biotinylated oligo(dT) primer is added to the solution of total RNA and used to hybridize to the 3′ poly(A) region of the mRNAs. The hybridization products are captured and washed at high stringency using streptavidin coupled to paramagnetic particles and a magnetic separation stand. The mRNA is eluted from the solid phase by the simple addition of ribonuclease-free deionized water. Other approaches do not require the prior isolation of total RNA. For example, uniform, superparamagnetic, polystyrene beads with oligo(dT) sequences covalently bound to the surface may be used to isolate mRNA directly by specific base pairing between the poly(A) residues of mRNA and the oligo(dT) sequences on the beads. Furthermore, the oligo(dT) sequence on the beads may also be used as a primer for the reverse transcriptase to subsequently synthesize the first strand of cDNA. Alternatively, new methods or improvements of existing methods for total RNA or mRNA isolation, preparation and purification may be devised by one skilled in the art and used in the practice of the methods of the invention.

Numerous different and versatile kits can be used to extract RNA (i.e., total RNA or mRNA) from bodily fluids and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), Dynal Biotech Inc. (Lake Success, N.Y.), Epicentre Technologies (Madison, Wis.), Gentra Systems, Inc. (Minneapolis, Minn.), GIBCO BRL (Gaithersburg, Md.), Invitrogen Life Technologies (Carlsbad, Calif.), MicroProbe Corp. (Bothell, Wash.), Organon Teknika (Durham, N.C.), Promega, Inc. (Madison, Wis.), and Qiagen Inc. (Valencia, Calif.). For example, the RNAprotect Saliva Kit (Qiagen) may be used to extract salivary RNA. User Guides that describe in great detail the protocol to be followed are usually included in all these kits. Sensitivity, processing time and cost may be different from one kit to another. One of ordinary skill in the art can easily select the kit(s) most appropriate for a particular situation.

### Amplification of Extracted Neonatal Saliva RNA

In certain embodiments, the saliva neonatal RNA is amplified before being analyzed. In some embodiments, before analysis, the saliva neonatal RNA is converted, by reverse-transcriptase, into complementary DNA (cDNA), which, optionally, may, in turn, be converted into complementary RNA (cRNA) by transcription.

Amplification methods are well known in the art (see, for example, Kimmel and Berger (1987), Sambrook et al (1989), Ausubel (Ed.) (2002), and U.S. Pat. Nos. 4,683,195; 4,683,202 and 4,800,159). Standard nucleic acid amplification methods include: polymerase chain reaction (or PCR, see, for example, Innis (Ed.) (1990) and Innis (Ed.) (1995)) and ligase chain reaction (or LCR, see, for example, Landegren et al. (1988); and Barringer (1990)).

Methods for transcribing RNA into cDNA are also well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase). Other methods include transcription-based amplification system (TAS) (see, for example, Kwoh et al. (1989)), isothermal transcription-based systems such as Self-Sustained Sequence Replication (3SR) (see, for example, Guatelli et al. (1990)), and Q-beta replicase amplification (see, for example, Smith et al. (1997); and Burg et al., (1996)).

The cDNA products resulting from these reverse transcriptase methods may serve as templates for multiple rounds of transcription by the appropriate RNA polymerase (for example, by nucleic acid sequence based amplification or NASBA, see, for example, Kievits et al. (1991), and Greijer et al. (2001)). Transcription of the cDNA template rapidly amplifies the signal from the original target mRNA.

In some embodiments, nucleic acid amplification methods designed to amplify from limited biological material (e.g., from a single cell) and/or from the entire transcriptome are used. (Amplification of the entire transcriptome may be particulrly desirable for global gene expression analyses.) For example, NuGEN Technologies's (www.nugeninc.com) RNA amplification systems are suitable for use in the practice of the invention and are described in U.S. Pat. Nos. 6,692,918; 6,251,639; 6,946,251 (the contents of which are herein incorporated by reference in their entirety). NuGEN amplification systems include, but are not limited to, WT-Ovation™ RNA Amplification System, WT-Ovation™ Pico RNA Amplification System, WT-Ovation™ FFPE System V2, and Ovation® RNA Amplification System V2. With NuGEN's Ribo-SPIA™ technology, amplification of target RNA molecules is initiated at both the 3′ end and randomly throughout the transcriptome using a first strand DNA/RNA chimeric primer mix and reverse transcriptase (RT). Microgram quantities of cDNA can be prepared from as little as 500 pg to 50 ng total RNA.

These methods as well as others (either known or newly devised by one skilled in the art) may be used in the practice of the invention.

Amplification can also be used to quantify the amount of extracted neonatal RNA (see, for example, U.S. Pat. No. 6,294,338). Alternatively or additionally, amplification using appropriate oligonucleotide primers can be used to label cell-free neonatal RNA prior to analysis (see below). Suitable oligonucleotide amplification primers can easily be selected and designed by one skilled in the art.

### Labeling of Neonatal Saliva RNA

In certain embodiments, neonatal saliva RNA (for example, after amplification, or after conversion to cDNA or to cRNA) is labeled with a detectable agent or moiety before being analyzed. The role of a detectable agent is to facilitate detection of neonatal RNA or to allow visualization of hybridized nucleic acid fragments (e.g., nucleic acid fragments bound to genetic probes). In some embodiments, the detectable agent is selected such that it generates a signal which can be measured and whose intensity is related to the amount of labeled nucleic acids present in the sample being analyzed. In array-based analysis methods, the detectable agent is also in some embodiments selected such that it generates a localized signal, thereby allowing spatial resolution of the signal from each spot on the array.

The association between the nucleic acid molecule and detectable agent can be covalent or non-covalent. Labeled nucleic acid fragments can be prepared by incorporation of or conjugation to a detectable moiety. Labels can be attached directly to the nucleic acid fragment or indirectly through a linker. Linkers or spacer arms of various lengths are known in the art and are commercially available, and can be selected to reduce steric hindrance, or to confer other useful or desired properties to the resulting labeled molecules (see, for example, Mansfield et al. (1995)).

Methods for labeling nucleic acid molecules are well-known in the art. For a review of labeling protocols, label detection techniques and recent developments in the field (see, for example, Kricka (2002), van Gijlswijk et al. (2001), and Joos et al. (1994)). Standard nucleic acid labeling methods include: incorporation of radioactive agents, direct attachment of fluorescent dyes (see, for example, Smith et al. (1985)) or of enzymes (see, for example, Connoly and Rider (1985)); chemical modifications of nucleic acid fragments making them detectable immunochemically or by other affinity reactions (see, for example, Broker et al. (1978), Bayer et al., (1980), Langer et al. (1981), Richardson et al. (1983), Brigati et al. (1983), Tchen et al. (1984), Landegent et al. (1984), and Hopman et al. (1987)); and enzyme-mediated labeling methods, such as random priming, nick translation, PCR and tailing with terminal transferase (for a review on enzymatic labeling, see, for example, Temsamani and Agrawal (1996)). More recently developed nucleic acid labeling systems include, but are not limited to: ULS (Universal Linkage System; see, for example, Wiegant et al. (1999)), photoreactive azido derivatives (see, for example, Neves et al. (2000)), and alkylating agents (see, for example, Sebestyen et al. (1998)).

Any of a wide variety of detectable agents can be used in the practice of the present invention. Suitable detectable agents include, but are not limited to: various ligands, radionuclides (such as, for example, ³²P, ³⁵S, ³H, ¹⁴C, ¹²⁵I, ¹³¹I and the like); fluorescent dyes (for specific exemplary fluorescent dyes, see below); chemiluminescent agents (such as, for example, acridinium esters, stabilized dioxetanes and the like); microparticles (such as, for example, quantum dots, nanocrystals, phosphors and the like); enzymes (such as, for example, those used in an ELISA, i.e., horseradish peroxidase, beta-galactosidase, luciferase, alkaline phosphatase); colorimetric labels (such as, for example, dyes, colloidal gold and the like); magnetic labels (such as, for example, Dynabeads™); and biotin, dioxigenin or other haptens and proteins for which antisera or monoclonal antibodies are available.

In certain embodiments, neonatal saliva RNA (after amplification, or conversion to cDNA or to cRNA) is fluorescently labeled. Numerous known fluorescent labeling moieties of a wide variety of chemical structures and physical characteristics are suitable for use in the practice of this invention. Suitable fluorescent dyes include, but are not limited to: Cy-3™, Cy-5™, Texas red, FITC, phycoerythrin, rhodamine, fluorescein, fluorescein isothiocyanine, carbocyanine, merocyanine, styryl dye, oxonol dye, BODIPY dye (i.e., boron dipyrromethene difluoride fluorophore, see, for example, Chen et al. (2000), Chen et al. (2000), U.S. Pat. Nos. 4,774,339; 5,187,288; 5,227,487; 5,248,782; 5,614,386; 5,994,063; and 6,060,324), and equivalents, analogues, derivatives or combinations of these molecules. Similarly, methods and materials are known for linking or incorporating fluorescent dyes to biomolecules such as nucleic acids (see, for example, Haugland (1994)). Fluorescent labeling dyes as well as labeling kits are commercially available from, for example, Amersham Biosciences, Inc. (Piscataway, N.J.), Molecular Probes, Inc. (Eugene, Oreg.), and New England Biolabs, Inc. (Beverly, Mass.).

Favorable properties of fluorescent labeling agents to be used in the practice of the invention include high molar absorption coefficient, high fluorescence quantum yield, and photostability. Some labeling fluorophores exhibit absorption and emission wavelengths in the visible (i.e., between 400 and 750 nm) rather than in the ultraviolet range of the spectrum (i.e., lower than 400 nm).

In other embodiments, neonatal saliva RNA (for example, after amplification or conversion to cDNA or cRNA) is made detectable through one of the many variations of the biotin-avidin system, which are well known in the art. Biotin RNA labeling kits are commercially available, for example, from Roche Applied Science (Indianapolis, Ind.) Perkin Elmer (Boston, Mass.), and NuGEN (San Carlos, Calif.).

Detectable moieties can also be biological molecules such as molecular beacons and aptamer beacons. Molecular beacons are nucleic acid molecules carrying a fluorophore and a non-fluorescent quencher on their 5′ and 3′ ends. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and allowing emission of a fluorescence signal. The use of molecular beacons as detectable moieties is well-known in the art (see, for example, Sokol et al. (1998); and U.S. Pat. Nos. 6,277,581 and 6,235,504). Aptamer beacons are similar to molecular beacons except that they can adopt two or more conformations (see, for example, Kaboev et al. (2000), Yamamoto et al. (2000), Hamaguchi et al. (2001), and Poddar and Le (2001)).

A “tail” of normal or modified nucleotides may also be added to nucleic acid fragments for detectability purposes. A second hybridization with nucleic acid complementary to the tail and containing a detectable label (such as, for example, a fluorophore, an enzyme or bases that have been radioactively labeled) allows visualization of the nucleic acid fragments bound to the array (see, for example, system commercially available from Enzo Biochem Inc., New York, N.Y.).

The selection of a particular nucleic acid labeling technique will depend on the situation and will be governed by several factors, such as the ease and cost of the labeling method, the quality of sample labeling desired, the effects of the detectable moiety on the hybridization reaction (e.g., on the rate and/or efficiency of the hybridization process), the nature of the detection system to be used, the nature and intensity of the signal generated by the detectable label, and the like.

II. Analysis of Neonatal RNA from Saliva

According to the present invention, neonatal saliva RNA can be analyzed to obtain information regarding the neonatal RNA. In certain embodiments, analyzing the neonatal saliva RNA comprises determining the quantity, concentration or sequence composition of neonatal RNA.

Neonatal saliva RNA may be analyzed by any of a variety of methods. Methods of analysis of RNA are well-known in the art (see, for example, Sambrook et al. (1989) and Ausubel (Ed.) (2002)).

For example, the quantity and concentration of neonatal RNA extracted from saliva may be evaluated by UV spectroscopy, wherein the absorbance of a diluted RNA sample is measured at 260 and 280 nm (Wilfinger et al. (1997)). Quantitative measurements may also be carried out using certain fluorescent dyes, such as, for example, RiboGreen® (commercially available from Molecular Probes, Eugene, Oreg.), which exhibit a large fluorescence enhancement when bound to nucleic acids. RNA labeled with these fluorescent dyes can be detected using standard fluorometers, fluorescence microplate reader or filter fluorometers. Another method for analyzing quantity and quality of RNA samples is through use of a BioAnalyzer (commercially available from Agilent Technologies, Foster City, Calif.), which separates charged biological molecules (such as nucleic acids) using microfluidic technologies and then a laser to excite intercalating fluorescent dyes.

Neonatal saliva RNA may also be analyzed through sequencing. For example, RNase T1, which cleaves single-stranded RNA specifically at the 3′-side of guanosine residues in a two-step mechanism, may be used to digest denatured RNA. Partial digestion of 3′ or 5′ labeled RNA with this enzyme thus generates a ladder of G residues. The cleavage can be monitored by radioactive (Ikehara et al. (1986)) and photometric (Grunert et al (1993)) detection systems, by zymogram assay (Bravo et al. (1994)), agar diffusion test (Quaas et al. (1989)), lanthan assay (Anfinsen et al. (1954)) or methylene blue test (Greiner-Stoeffele et al. (1996)) or by fluorescence correlation spectroscopy (Korn et al. (2000)).

Other methods for analyzing neonatal saliva RNA include northern blots, wherein the components of the RNA sample being analyzed are resolved by size prior to detection thereby allowing identification of more than one species simultaneously, and slot/dot blots, wherein unresolved mixtures are used.

In certain embodiments, analyzing the neonatal saliva RNA comprises submitting the extracted RNA to a gene-expression analysis. In some embodiments, this includes the simultaneous analysis of multiple genes, such as genes known or discovered to be involved in a particular disease or condition, and/or in neonatal development (and particularly in neonatal feeding characteristics).

Some examples of such genes include, but are not limited to: nuclear factor kappa B (NFκB), I kappa B-alpha (IκB-α), toll-like receptor 4 (TLR4), platelet activating factor (PAF), platelet activating factor acetylhydrolase (PAF-AH), interleukin 8 (IL-8), epidermal growth factor (EGF), interleukin 10 (IL-10), endothelial 1 (ET-1), and combinations thereof. Additional genes are described herein.

As another example, analysis of neonatal saliva RNA may include detection of the presence of and/or quantitating RNA transcribed from genes that are involved in feeding and digestion. These include genes encoding digestive enzymes such as luminal enterokinase, lactase, carboyxpeptidase D., etc.

Analysis of neonatal saliva RNA may include detection of the presence of RNA transcribed from mesenchymal developmental genes, neurodevelopmental genes, cytokines, and immunoglobulins. These genes include neurturin, glial cell derived neurotrophic factor, B-cell CLL/Lymphoma 2, etc. As another example, detection of and/or determining expression levels of surfactant genes may be used as a way of monitoring neonatal lung development.

In analyses carried out to detect the presence or absence of RNA transcribed from a specific gene, the detection may be performed by any of a variety of physical, immunological and biochemical methods. Such methods are well-known in the art, and include, for example, protection from enzymatic degradation such as S1 analysis and RNase protection assays, in which hybridization to a labeled nucleic acid probe is followed by enzymatic degradation of single-stranded regions of the probe and analysis of the amount and length of probe protected from degradation.

In some embodiments of the invention, real time RT-PCR, methods are employed that allow quantification of RNA transcripts and viewing of the increase in amount of nucleic acid as it is amplified. The TaqMan assay, a quenched fluorescent dye system, may also be used to quantitate targeted mRNA levels (see, for example Livak et al. (1995)).

In some embodiments of the invention involving methods that allow quantification of RNA transcripts (such as real time RT-PCR) expression, reference genes are used as normalization controls. Examples of reference genes include GAPDH, 18S rRNA, beta-actin, cyclophilin, tubulin, etc.

In some embodiments of the invention involving methods (such as real time RT-PCR) that allow quantification of particular RNA transcript expression, amplification is performed for multiple reference genes; in some such embodiments, such reference genes maintain a relative constant and consistent range of expression across different post-conceptual ages (PCAs). Particular examples of such reference genes that may be utilized in accordance with the present invention include glyceraldehyde-3-phosphate dehydrogenase (GAPDH), tyrosine 3-monoxygenase/tryptophan 5-monooxygenase activation protein, zeta polypeptide (YWHAZ), hypoxanthine phosphoribosyltransferase 1 (HPRT1), and combinations thereof.

Other methods are based on the analysis of cDNA derived from mRNA, which is less sensitive to degradation than RNA and therefore easier to handle. These methods include, but are not limited to, sequencing cDNA inserts of an expressed sequence tag (EST) clone library (see, for example, Adams et al. (1991)) and serial analysis of gene expression (or SAGE), which allows quantitative and simultaneous analysis of a large number of transcripts (see, for example, U.S. Pat. No. 5,866,330; Velculescu et al. (1995); and Zhang et al. (1997)). These two methods survey the whole spectrum of mRNA in a sample rather than focusing on a predetermined set.

Other methods of analysis of cDNA derived from mRNA include reverse transcriptase-mediated PCR(RT-PCR) gene expression assays. These methods are directed at specific target gene products and allow the qualitative (non-quantitative) detection of transcripts of very low abundance (see, for example, Su et al. (1997)). A variation of these methods, called competitive RT-PCR, in which a known amount of exogenous template is added as internal control, has been developed to allow quantitative measurements (see, for example, Becker-Andre and Hahlbrock (1989), Wang (1989), and Gilliland et al. (1990)).

In some embodiments of the present invention, multiplex quantitative real-time PCR (qRT-PCR) is performed to produce amplicons specific to DNA (gene) sequences (see, for example, Hayden et al. (2008)). In some such embodiments, multiplex RT-PCR is used to produce amplicons of a plurality of different gene sequences. In some particular embodiments, amplicons of different gene sequences are readily distinguishable from one another, for example based on size, extent of hybridization to a particular probe, etc.

mRNA analysis may also be performed by differential display reverse transcriptase PCR (DDRT-PCR; see, for example, Liang and Pardee (1992)) or RNA arbitrarily primed PCR (RAP-CPR; see, for example, Welsh et al. (1992) and McClelland et al. (1993)). In these methods, RT-PCR fingerprint profiles of transcripts are generated by random priming and differentially expressed genes appear as changes in the fingerprint profiles between two samples. Identification of a differentially expressed gene requires further manipulation (i.e., the appropriate band of the gel must be excised, subcloned, sequenced and matched to a gene in a sequence database).

Additional methods include sequencing-based strategies, such as Next Generation Sequencing (NGS), including RNASeq. Such strategies are known in the art (see, e.g., Cloonan (2008) and Tarazona (2011)). In some embodiments, RNASeq is used to analyze a salivary sample (for example, that has a volume below about 10 μL, 9 μL, 8 μL, 7 μL, 6 μL, 5 μL, 4 μL, 3 μL, 2 μL, or 1 μL, or less), to obtain whole transcriptome sequencing. Such analysis can be used to identify genes that are differentially expressed and/or differentially regulated in neonates, such as neonates exhibiting impaired neonatal development or neonatal feeding characteristic.

### III. Array-Based Gene Expression Analysis of Neonatal Saliva RNA

In certain embodiments, the methods of the invention include submitting neonatal saliva RNA to an array-based gene expression analysis.

### Array-Based Gene Expression Analysis

Traditional molecular biology methods, such as most of those described above, typically assess one gene per experiment, which significantly limits the overall throughput and prevents gaining a broad picture of gene function. Technologies based on DNA array or microarray (also called gene expression microarray), which were developed more recently, offer the advantage of allowing the monitoring of thousands of genes simultaneously through identification of sequence (gene/gene mutation) and determination of gene expression level (abundance) of genes (see, for example, Marshall and Hodgson (1998), Ramsay, (1998), Ekins and Chu (1999), and Lockhart and Winzeler (2000)).

In a gene expression experiment, labeled cDNA or cRNA targets derived from the mRNA of an experimental sample are hybridized to nucleic acid probes immobilized to a solid support. By monitoring the amount of label associated with each DNA location, it is possible to infer the abundance of each mRNA species represented.

There are two standard types of DNA microarray technology in terms of the nature of the arrayed DNA sequence. In the first format, probe cDNA sequences (typically 500 to 5,000 bases long) are immobilized to a solid surface and exposed to a plurality of targets either separately or in a mixture. In the second format, oligonucleotides (typically 20-80-mer oligos) or peptide nucleic acid (PNA) probes are synthesized either in situ (i.e., directly on-chip) or by conventional synthesis followed by on-chip attachment, and then exposed to labeled samples of nucleic acids.

The analyzing step in the methods of the invention can be performed using any of a variety of methods, means and variations thereof for carrying out array-based gene expression analysis. Array-based gene expression methods are known in the art and have been described in numerous scientific publications as well as in patents (see, for example, Schena et al. (1995), Schena et al. (1996), and Chen et al. (1998); U.S. Pat. Nos. 5,143,854; 5,445,934; 5,807,522; 5,837,832; 6,040,138; 6,045,996; 6,284,460; and 6,607,885).

Additional array-based methods include qRT-PCR arrays, including high-throughput mid-density qRT-PCR arrays. Such arrays are known in the art and commercially available (for example, OpenArray® from Applied Biosystems).

In the practice of the present invention, these methods as well as other methods known in the art for carrying out array-based gene expression analysis may be used as described or modified such that they allow neonatal mRNA levels of gene expression to be evaluated.

### Test Sample

In some embodiments, neonatal saliva RNA to be analyzed in accordance with the present invention is isolated from a sample of saliva as described above. A test sample of neonatal saliva RNA to be used in the methods of the invention may include a plurality of nucleic acid fragments labeled with a detectable agent.

In some embodiments, neonatal saliva RNA is isolated from a saliva sample that has a volume below about 50 μL, 45 μL, 40 μL, 35 μL, 30 μL, 25 μL, 20 μL, or even less. In some embodiments neonatal saliva RNA is isolated from a saliva sample that has a volume below about 10 μL, 9 μL, 8 μL, 7 μL, 6 μL, 5 μL, 4 μL, 3 μL, 2 μL, or even 1 μL or less.

Extracted neonatal RNA may be amplified, reverse-transcribed, labeled, fragmented, purified, concentrated and/or otherwise modified prior to the gene-expression analysis. Techniques for the manipulation of nucleic acids are well-known in the art, see, for example, Sambrook et al., (1989), Innis (Ed.) (1990), Tijssen (1993), Innis (Ed.) (1995), and Ausubel (Ed.) (2002).

In certain embodiments, in order to improve the resolution of the array-based gene expression analysis, the nucleic acid fragments of the test sample are less then 500 bases long, in some embodiments less than about 200 bases long. The use of small fragments significantly increases the reliability of the detection of small differences or the detection of unique sequences.

Methods of RNA fragmentation are known in the art and include: treatment with ribonucleases (e.g., RNase T1, RNase V1 and RNase A), sonication (see, for example, Deininger (1983)), mechanical shearing, and the like (see, for example, Sambrook et al. (1989), Tijssen (1993), Ordahl et al. (1976), Oefner et al. (1996), Thorstenson et al. (1998)). Random enzymatic digestion of the RNA leads to fragments containing as low as 25 to 30 bases.

Fragment size of the nucleic acid segments in the test sample may be evaluated by any of a variety of techniques, such as, for example, electrophoresis (see, for example, Siles and Collier (1997)) or matrix-assisted laser desorption/ionization time-of-flight mass spectrometry (see, for example, Chiu et al. (2000)).

In the practice of certain methods of the invention, the test sample of neonatal saliva RNA is labeled before analysis. Suitable methods of nucleic acid labeling with detectable agents have been described in detail above.

Prior to hybridization, the labeled nucleic acid fragments of the test sample may be purified and concentrated before being resuspended in the hybridization buffer. Columns such as Microcon 30 columns may be used to purify and concentrate samples in a single step. Alternatively or additionally, nucleic acids may be purified using a membrane column (such as a Qiagen column) or Sephadex G50 and precipitated in the presence of ethanol.

### Gene-Expression Hybridization Arrays

Any of a variety of arrays may be used in the practice of the present invention. Investigators can either rely on commercially available arrays or generate their own. Methods of making and using arrays are well known in the art (see, for example, Kern and Hampton, (1997), Schummer et al., (1997), Solinas-Toldo et al. (1997), Johnston (1998), Bowtell (1999), Watson and Akil (199), Freeman et al. (2000), Lockhart and Winzeler (2000), Cuzin (2001), Zarrinkar et al., (2001), Gabig and Wegrzyn, (2001), and Cheung et al. (2001); see also, for example, U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893).

Arrays comprise a plurality of genetic probes immobilized to discrete spots (i.e., defined locations or assigned positions) on a substrate surface. Gene arrays used in accordance with some embodiments of the invention contain probes representing a comprehensive set of genes across the genome. In some such embodiments, the genes represented by the probes do not represent any particular subset of genes, and/or may be a random assortment of genes. In some embodiments of the invention, the gene arrays comprise a particular subset or subsets of genes. The subsets of genes may represent particular classes of genes of interest. For example, an array comprising probes for developmental genes may be used in order to focus analyses on developmental genes. In such embodiments using arrays having particular subsets, more than one class of genes of interest may be represented on the same array.

Substrate surfaces suitable for use in the present invention can be made of any of a variety of rigid, semi-rigid or flexible materials that allow direct or indirect attachment (i.e., immobilization) of genetic probes to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may in some embodiments be used (see, for example, U.S. Pat. No. 6,063,338).

The presence of reactive functional chemical groups (such as, for example, hydroxyl, carboxyl, amino groups and the like) on the material can be exploited to directly or indirectly attach genetic probes to the substrate surface. Methods for immobilizing genetic probes to substrate surfaces to form an array are well-known in the art.

More than one copy of each genetic probe may be spotted on the array (for example, in duplicate or in triplicate). This arrangement may, for example, allow assessment of the reproducibility of the results obtained. Related genetic probes may also be grouped in probe elements on an array. For example, a probe element may include a plurality of related genetic probes of different lengths but comprising substantially the same sequence. Alternatively, a probe element may include a plurality of related genetic probes that are fragments of different lengths resulting from digestion of more than one copy of a cloned piece of DNA. A probe element may also include a plurality of related genetic probes that are identical fragments except for the presence of a single base pair mismatch. An array may contain a plurality of probe elements. Probe elements on an array may be arranged on the substrate surface at different densities.

Array-immobilized genetic probes may be nucleic acids that contain sequences from genes (e.g., from a genomic library), including, for example, sequences that collectively cover a substantially complete genome or a subset of a genome (for example, the array may contain only human genes that are expressed throughout development). Genetic probes may be long cDNA sequences (500 to 5,000 bases long) or shorter sequences (for example, 20-80-mer oligonucleotides). The sequences of the genetic probes are those for which gene expression levels information is desired. Additionally or alternatively, the array may comprise nucleic acid sequences of unknown significance or location. Genetic probes may be used as positive or negative controls (for example, the nucleic acid sequences may be derived from karyotypically normal genomes or from genomes containing one or more chromosomal abnormalities; alternatively or additionally, the array may contain perfect match sequences as well as single base pair mismatch sequences to adjust for non-specific hybridization).

Techniques for the preparation and manipulation of genetic probes are well-known in the art (see, for example, Sambrook et al. (1989), Innis (Ed.) (1990), Tijssen (1993), Innis (Ed.) (1995), and Ausubel (Ed.) (2002)).

Long cDNA sequences may be obtained and manipulated by cloning into various vehicles. They may be screened and re-cloned or amplified from any source of genomic DNA. Genetic probes may be derived from genomic clones including mammalian and human artificial chromosomes (MACs and HACs, respectively, which can contain inserts from ˜5 to 400 kilobases (kb)), satellite artificial chromosomes or satellite DNA-based artificial chromosomes (SATACs), yeast artificial chromosomes (YACs; 0.2-1 Mb in size), bacterial artificial chromosomes (BACs; up to 300 kb); P1 artificial chromosomes (PACs; ˜70-100 kb) and the like.

Genetic probes may also be obtained and manipulated by cloning into other cloning vehicles such as, for example, recombinant viruses, cosmids, or plasmids (see, for example, U.S. Pat. Nos. 5,266,489; 5,288,641 and 5,501,979).

In some embodiments, genetic probes are synthesized in vitro by chemical techniques well-known in the art and then immobilized on arrays. Such methods are especially suitable for obtaining genetic probes comprising short sequences such as oligonucleotides and have been described in scientific articles as well as in patents (see, for example, Narang et al. (1979), Brown et al. (1979), Belousov et al. (1997), Guschin et al. (1997), Blommers et al., (1994) and Frenkel et al. (1995); see also for example, U.S. Pat. No. 4,458,066).

For example, oligonucleotides may be prepared using an automated, solid-phase procedure based on the phosphoramidite approach. In such a method, each nucleotide is individually added to the 5-end of the growing oligonucleotide chain, which is attached at the 3′-end to a solid support. The added nucleotides are in the form of trivalent 3′-phosphoramidites that are protected from polymerization by a dimethoxytrityl (or DMT) group at the 5-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on commercial oligo synthesizers such as the Perkin Elmer/Applied Biosystems Division DNA synthesizer.

Methods of attachment (or immobilization) of oligonucleotides on substrate supports have been described (see, for example, Maskos and Southern (1992), Matson et al. (1995), Lipshutz et al. (1999), Rogers et al. (1999), Podyminogin et al. (2001), Belosludtsev et al. (2001)).

Oligonucleotide-based arrays have also been prepared by synthesis in situ using a combination of photolithography and oligonucleotide chemistry (see, for example, Pease et al. (1994), Lockhart et al. (1996), Singh-Gasson et al. (1999), Pirrung et al. (2001), McGall et al., (2001), Barone et al. (2001), Butler et al. (2001), Nuwaysir et al. (2002)). The chemistry for light-directed oligonucleotide synthesis using photolabile protected 2′-deoxynucleoside phosphoramites has been developed by Affymetrix Inc. (Santa Clara, Calif.) and is well known in the art (see, for example, U.S. Pat. Nos. 5,424,186 and 6,582,908).

An alternative to custom arraying of genetic probes is to rely on commercially available arrays and micro-arrays. Such arrays have been developed, for example, by Affymetrix Inc. (Santa Clara, Calif.), Illumina, Inc. (San Diego, Calif.), Spectral Genomics, Inc. (Houston, Tex.), and Vysis Corporation (Downers Grove, Ill.).

### Hybridization

In certain methods of the invention, a gene expression array may be contacted with the test sample under conditions wherein nucleic acid fragments in the sample specifically hybridize to genetic probes immobilized on the array.

Hybridization reaction and washing step(s), if any, may be carried out under any of a variety of experimental conditions. Numerous hybridization and wash protocols have been described and are well-known in the art (see, for example, Sambrook et al. (1989), Tijssen (1993), Innis (Ed.) (1995), and Anderson (Ed.) (1999)). The methods of the invention may be carried out by following known hybridization protocols, by using modified or optimized versions of known hybridization protocols or newly developed hybridization protocols as long as these protocols allow specific hybridization to take place.

The term “specific hybridization” refers to a process in which a nucleic acid molecule preferentially binds, duplexes, or hybridizes to a particular nucleic acid sequence under stringent conditions. In the context of the present invention, this term more specifically refers to a process in which a nucleic acid fragment from a test sample preferentially binds (i.e., hybridizes) to a particular genetic probe immobilized on the array and to a lesser extent, or not at all, to other immobilized genetic probes of the array. Stringent hybridization conditions are sequence dependent. The specificity of hybridization increases with the stringency of the hybridization conditions; reducing the stringency of the hybridization conditions results in a higher degree of mismatch being tolerated.

The hybridization and/or wash conditions may be adjusted by varying different factors such as the hybridization reaction time, the time of the washing step(s), the temperature of the hybridization reaction and/or of the washing process, the components of the hybridization and/or wash buffers, the concentrations of these components as well as the pH and ionic strength of the hybridization and/or wash buffers.

In certain embodiments, the hybridization and/or wash steps are carried out under very stringent conditions. In other embodiments, the hybridization and/or wash steps are carried out under moderate to stringent conditions. In still other embodiments, more than one washing steps are performed. For example, in order to reduce background signal, a medium to low stringency wash is followed by a wash carried out under very stringent conditions.

As is well known in the art, the hybridization process may be enhanced by modifying other reaction conditions. For example, the efficiency of hybridization (i.e., time to equilibrium) may be enhanced by using reaction conditions that include temperature fluctuations (i.e., differences in temperature that are higher than a couple of degrees). An oven or other devices capable of generating variations in temperatures may be used in the practice of the methods of the invention to obtain temperature fluctuation conditions during the hybridization process.

It is also known in the art that hybridization efficiency is significantly improved if the reaction takes place in an environment where the humidity is not saturated. Controlling the humidity during the hybridization process provides another means to increase the hybridization sensitivity. Array-based instruments usually include housings allowing control of the humidity during all the different stages of the experiment (i.e., pre-hybridization, hybridization, wash and detection steps).

Additionally or alternatively, a hybridization environment that includes osmotic fluctuation may be used to increase hybridization efficiency. Such an environment where the hyper-/hypo-tonicity of the hybridization reaction mixture varies may be obtained by creating a solute gradient in the hybridization chamber, for example, by placing a hybridization buffer containing a low salt concentration on one side of the chamber and a hybridization buffer containing a higher salt concentration on the other side of the chamber

### Highly Repetitive Sequences

In the practice of the methods of the invention, an array may be contacted with the a test sample under conditions wherein nucleic acid segments in the sample specifically hybridize to genetic probes on the array. As mentioned above, the selection of appropriate hybridization conditions allows specific hybridization to take place. In certain cases, the specificity of hybridization may further be enhanced by inhibiting repetitive sequences.

In certain embodiments, repetitive sequences present in the nucleic acid fragments are removed or their hybridization capacity is disabled. By excluding repetitive sequences from the hybridization reaction or by suppressing their hybridization capacity, one prevents the signal from hybridized nucleic acids to be dominated by the signal originating from these repetitive-type sequences (which are statistically more likely to undergo hybridization). Failure to remove repetitive sequences from the hybridization or to suppress their hybridization capacity results in non-specific hybridization, making it difficult to distinguish the signal from the background noise.

Removing repetitive sequences from a mixture or disabling their hybridization capacity can be accomplished using any of a variety of methods well-known to those skilled in the art. These methods include, but are not limited to, removing repetitive sequences by hybridization to specific nucleic acid sequences immobilized to a solid support (see, for example, Brison et al. (1982)); suppressing the production of repetitive sequences by PCR amplification using adequate PCR primers; or inhibiting the hybridization capacity of highly repeated sequences by self-reassociation (see, for example, Britten et al. (1974)).

In some embodiments, the hybridization capacity of highly repeated sequences is competitively inhibited by including, in the hybridization mixture, unlabeled blocking nucleic acids. The unlabeled blocking nucleic acids, which are mixed to the test sample before the contacting step, act as a competitor and prevent the labeled repetitive sequences from binding to the highly repetitive sequences of the genetic probes, thus decreasing hybridization background. In certain embodiments, for example when cDNA derived from neonatal mRNA is analyzed, the unlabeled blocking nucleic acids are Human Cot-1 DNA. Human Cot-1 DNA is commercially available, for example, from Gibco/BRL Life Technologies (Gaithersburg, Md.).

### Binding Detection and Data Analysis

In some embodiments, inventive methods include determining the binding of individual nucleic acid fragments of the test sample to individual genetic probes immobilized on the array in order to obtain a binding pattern. In array-based gene expression, determination of the binding pattern is carried out by analyzing the labeled array that results from hybridization of labeled nucleic acid segments to immobilized genetic probes.

In certain embodiments, determination of the binding includes: measuring the intensity of the signals produced by the detectable agent at each discrete spot on the array.

Analysis of the labeled array may be carried out using any of a variety of means and methods, whose selection will depend on the nature of the detectable agent and the detection system of the array-based instrument used.

In certain embodiments, the detectable agent comprises a fluorescent dye and the binding is detected by fluorescence. In other embodiments, the sample of neonatal saliva RNA is biotin-labeled and after hybridization to immobilized genetic probes, the hybridization products are stained with a streptavidin-phycoerythrin conjugate and visualized by fluorescence. Analysis of a fluorescently labeled array usually comprises: detection of fluorescence over the whole array, image acquisition, quantitation of fluorescence intensity from the imaged array, and data analysis.

Methods for the detection of fluorescent labels and the creation of fluorescence images are well known in the art and include the use of “array reading” or “scanning” systems, such as charge-coupled devices (i.e., CCDs). Any known device or method, or variation thereof can be used or adapted to practice the methods of the invention (see, for example, Hiraoka et al., (1987), Aikens et al. (1989), Divane et al. (1994), Jalal et al. (1998), and Cheung et al. (1999); see also, for example, U.S. Pat. Nos. 5,539,517; 5,790,727; 5,846,708; 5,880,473; 5,922,617; 5,943,129; 6,049,380; 6,054,279; 6,055,325; 6,066,459; 6,140,044; 6,143,495; 6,191,425; 6,252,664; 6,261,776 and 6,294,331).

Commercially available microarrays scanners are typically laser-based scanning systems that can acquire one (or more) fluorescent image (such as, for example, the instruments commercially available from PerkinElmer Life and Analytical Sciences, Inc. (Boston, Mass.), Virtek Vision, Inc. (Ontario, Canada) and Axon Instruments, Inc. (Union City, Calif.)). Arrays can be scanned using different laser intensities in order to ensure the detection of weak fluorescence signals and the linearity of the signal response at each spot on the array. Fluorochrome-specific optical filters may be used during the acquisition of the fluorescent images. Filter sets are commercially available, for example, from Chroma Technology Corp. (Rockingham, Vt.).

In some embodiments, a computer-assisted imaging system capable of generating and acquiring fluorescence images from arrays such as those described above, is used in the practice of the methods of the invention. One or more fluorescent images of the labeled array after hybridization may be acquired and stored.

In some embodiments, a computer-assisted image analysis system is used to analyze the acquired fluorescent images. Such systems allow for an accurate quantitation of the intensity differences and for an easier interpretation of the results. A software for fluorescence quantitation and fluorescence ratio determination at discrete spots on an array is usually included with the scanner hardware. Softwares and/or hardwares are commercially available and may be obtained from, for example, BioDiscovery (El Segundo, Calif.), Imaging Research (Ontario, Canada), Affymetrix, Inc. (Santa Clara, Calif.), Applied Spectral Imaging Inc. (Carlsbad, Calif.); Chroma Technology Corp. (Brattleboro, Vt.); Leica Microsystems, (Bannockburn, Ill.); and Vysis Inc. (Downers Grove, Ill.). Other softwares are publicly available (e.g., MicroArray Image Analysis, and Combined Expression Data and Sequence Analysis (http://rana.lbl.gov); Chiang et al. (2001); a system written in R and available through the Bioconductor project (www.bioconductor.org); a Java-based TM4 software system available from the Institute for Genomic Research (http://www.tigr.org/software); and a Web-based system developed at Lund University (base.thep.lu.se)).

Accurate determination of fluorescence intensities often requires normalization and determination of the fluorescence ratio baseline (Brazma and Vilo (2000)). Data reproducibility may be assessed by using arrays on which genetic probes are spotted in duplicate or triplicate. Baseline thresholds may also be determined using global normalization approaches (M. K. Kerr et al. (2000)). Other arrays include a set of maintenance genes which shows consistent levels of expression over a wide variety of tissues and allows the normalization and scaling of array experiments.

In the practice of the methods of the invention, any of a large variety of bioinformatics and statistical methods may be used to analyze data obtained by array-based gene expression analysis. Such methods are well known in the art (for a review of essential elements of data acquisition, data processing, data analysis, data mining and of the quality, relevance and validation of information extracted by different bioinformatics and statistical methods, see, for example, Watson et al. (1998), Duggan et al. (1999), Bassett et al. (1999), Hess et al. (2001), Marcotte and Date (2001), Weinstein et al. (2002), Dewey (2002), Butte (2002), Tamames et al. (2002), Xiang et al. (2003).

### IV. Gene Expression Patterns and Neonatal Health and Disease

### Methods of Detecting or Identifying Genes

In certain aspects, the invention provides methods of detecting or identifying genes of interest in neonatal health and disease, and particularly in neonatal feeding characteristics. Provided methods include methods for detecting or identifying genes involved in neonatal development, and particularly in neonatal feeding characteristics. Such methods comprise providing a neonatal saliva RNA sample, identifying differentially expressed genes (as compared to appropriate control samples), and determining that the differentially expressed genes are involved in neonatal development, and particularly in neonatal feeding characteristics.

Also provided are methods for detecting identifying genes involved in a condition or disease affecting neonates, and particularly in neonatal feeding characteristics. Such methods comprise providing a neonatal saliva RNA sample, identifying differentially expressed genes (as compared to appropriate control samples, such as from neonates not diagnosed with the condition or disease), and determining that the differentially expressed genes are involved in the condition or disease or disease, and particularly in neonatal feeding characteristics.

### Identifying Differentially Expressed Genes

A variety of methods of detecting gene expression have been described herein. Differentially expressed genes are genes whose expression level differs depending on the cell, tissue, and/or sample from which the gene products are obtained. Genes may be identified as differentially expressed through gene expression array experiments using microarrays. Such methods have been described herein and are also described in Examples 2-4. In such experiments, genes are identified as differentially expressed in comparison with a control. The choice of an appropriate control depends on what kinds of genes one would like to identify.

To detect or identify genes involved in neonatal development, and particularly in neonatal feeding characteristics, for example, one may compare gene expression data from test samples with data from control samples obtained from neonates who are at a different developmental stage and/or otherwise have different feeding characteristics than neonates from whom the test samples were obtained. As will be understood by those of skill in the art, a variety of criteria may be used in determining developmental stage and/or presence of particular feeding characteristics. In some embodiments, a control sample is obtained from a neonate having a normal feeding characteristic relative to a neonate from whom a test sample is obtained.

In some embodiments of the invention, developmental stage and/or feeding characteristics is/are assessed with respect to factors such as body weight. In some embodiments of the invention, developmental stage and/or feeding characteristics is/are assessed with respect to feeding capabilities, e.g., readiness to feed and/or feeding tolerance. In some embodiments of the invention, developmental stage and/or feeding characteristics is/are assessed with respect to gestational age. In some embodiments of the invention, developmental stage and/or feeding characteristics is/are assessed with respect to post-conceptual age. In some embodiments of the invention, developmental stage and/or feeding characteristics is/are assessed with respect to capability of breathing without assistance, coordination of breathing rhythms, etc. In some embodiments of the invention, developmental stage and/or feeding characteristics is/are assessed with respect to a combination of factors, including combinations of any of the afore-mentioned factors. As another example, to detect or identify genes involved in a condition or disease affecting neonates, and particularly to detect or identify genes involved in feeding characteristics, one may compare gene expression data from a cohort of neonates suffering from or diagnosed with a condition (e.g., delay of readiness to feed) with data from a cohort of neonates who do not suffer from or are diagnosed with that condition.

Methods of determining levels of gene expression have already been described herein. In gene expression array experiments, quantitative readouts of expression levels are typically provided. Typically, after normalization of data, genes having at least a 1.5-fold differences (i.e. a ratio of about 1.5) in expression levels between test and control samples may be considered “differentially expressed.” In some embodiments of the invention, genes considered to be differentially expressed show at least two-fold, at least five-fold, at least ten-fold, at least 15-fold, at least 20-fold, or at least 25-fold different expression levels compared to controls. (It is to be understood that the fold different expression levels can be determined in either direction, i.e., the expression levels for the test sample may be at least 1.5-fold higher or 1.5-fold lower than expression levels for the control sample.)

It will be appreciated, however, that both the fold-difference cutoff for being considered differentially expressed varies depending on several factors which may include, for example, the type of samples used, the quantity and quality of the RNA sample, the power of the statistical analyses, the type of genes of interest, etc. In some embodiments, a lower cutoff ratio (i.e.—fold difference) is used, e.g., ratios of about 1.4, or about 1.37. In some embodiments, a higher cutoff ratio than about 1.5 is used, e.g., about 2.5, about 3.0, about 3.5, about 4.0, about 4.5, about 5.0, etc.

In some embodiments of the invention, a preliminary list of genes is identified as being differentially expressed using a particular statistical method or particular set of experimental data. In some embodiments, the preliminary list is narrowed down. That is, genes are identified within the preliminary list. Determining which genes among the preliminary list may be done in a hypothesis-driven manner. For example, only genes on the preliminary list that are deemed to be physiologically relevant (as determined, by example, by what is known of the gene's function, localization, structure, etc.) may be ultimately identified as differentially expressed genes of interest. In some embodiments, genes are identified within the preliminary list without regard to a particular hypothesis. A subset of genes from the preliminary list may be identified as genes of interest using, for example, a different method of gene expression analysis, a different set of samples etc. In some embodiments, no further selection or identification of genes is done after obtaining the preliminary list of genes.

It will be understood that inventive methods may identify some genes that are not known, not previously described in the literature, and/or not catalogued in publicly available databases. For example, some gene expression microarrays may contain probes for genes that have not yet been characterized or known in the literature. In cases in which uncharacterized genes are identified as being differentially regulated, the genes may still be described as being “identified” because there is usually an identifier, e.g., a probe with a known sequence on the microarray that can be associated with the gene, a name of an expressed sequence tag, etc.

Determining that Genes are Involved in Development or in a Condition or Disease

In some embodiments, determining that the genes identified as being differentially expressed are involved in the developmental process, condition, or disease of interest comprises deciding that genes meeting a particular cutoff for differential expression are involved. In some embodiments, determining that the genes are involved comprises one or more further steps. These further steps may involve alternative methods to determine gene expression such as those described herein, assessment of the gene's function, etc. Assessment of the gene's function may involve any or a any combination of analyzing literature on the gene, analyzing information on the gene in gene databases (e.g., OMIM, www.ncbi.nlm.nih.gov/sites/entrez?db=OMIM; PubMed, www.ncbi.nlm.nih.gov/sites/entrez; NetAffx, www.affymetrix.comianalysis/index.affx; UniGene, www.ncbi.nlm.nih.gov.sites/entrez?db=unigene; Ingenuity®, www.ingenuity.com etc.), performing additional experiments that may elucidate the gene's function, (e.g., genetic, biochemical, structural, etc.) etc.

### Methods of Diagnosing

In some aspects, the invention provides methods of determining a diagnosis of a neonate. Such methods comprise steps of providing a sample of saliva RNA obtained from the neonate; detecting expression of at least one gene identified as being differentially expressed using other methods of the invention, and determining, based on the detected expression of the at least one gene, a diagnosis of the neonate.

### Neuropeptide Y2 Receptor (NPY2R)

As used herein, the term neuropeptide Y (NPY) receptors refers to the family of Gi/o protein-coupled receptors that are primarily expressed in the arcuate nucleus of the hypothalamus. NPY2R may also be found in tissues, including trabecular bone, vascular, colonic mucosa (see, for example, Yoo et al. (2011), Shi et al. (2010), Uddman et al. (2002), and Wang et al. (2010)). The NPY family consists of five receptors, which are known to be associated with hypothalamic regulation of feeding behavior, metabolism, and energy homeostasis in both rodents and humans (see, for example, Lin et al. (2004), Huang et al. (2008), and Butler et al. (2001)). Knock-out studies in mice of the neuropeptide Y2 receptor gene, NPY2R, in particular exhibit hyperhagia and excessive weight gain (see, for example, Naveilhan et al. (1999)). For example, a particular NPY2R useful in certain methods described herein is encoded by the nucleotide sequence of SEQ ID NO.: 1. In some embodiments of the present invention, NPY2R expression is assessed in neonatal saliva. The present invention encompasses the finding that NPY2R expression is highly predictive of an immature feeding pattern. Some of these observations have been described in Example 5. Without wishing to be bound by any particular theory, the present inventors suggest that NPY2R may play a critical role in neonatal feeding behavior, and that its expression may be down-regulated prior to successful oral feeding. The present invention provides various technologies and methodologies for detecting and/or quantifying NPY2R, for example in neonatal subjects whose feeding behavior is to be assessed.

Among other things, the present invention encompasses the finding that expression of NPY2R is independent of the presence of enteral nutrition if given by catherer. Without wishing to be bound by any particular theory, the present inventors propose that this finding suggests that stimulation of gastrointestinal tract alone may not be enough to cause decreased gene expression, and that down-regulation of NPY2R is observed only when infants are able to take at least some feeds by mouth. Thus, for example, the present invention encompasses methods of assessing NPY2R expression independent of presence or degree of GI tract stimulation.

Still further, the present invention encompasses the finding that although NPY2R expression is statistically significantly negatively correlated with advancing PCA, PCA of the newborn is not the only regulator of NPY2R expression. Without wishing to be bound by any particular theory, the present inventors note that this finding suggests that though advancing gestational age correlates with decreased NPY2R expression, the relationship may be complex and not mutually inclusive. Thus, for example, the present invention encompasses methods of assessing NPY2R expression at various PCA. The present invention specifically encompasses the finding that salivary gene expression has the potential to monitor regulation of feeding behavior.

### Other Genes

Products of genes identified in other inventive methods may be used as markers in diagnostic methods in accordance with the present invention. Some potentially appropriate genes have been identified, for example, by experiments described in Example 2. In some embodiments of the invention, expression of one or more genes selected from the group consisting of glutamate-cysteine ligase, catalytic subunit, CD3d, cholecytokinin A receptor, fibroblast growth receptor 2, arginase liver and combinations thereof is detected and/or identified. In some embodiments, expression of one or more genes upregulated during neonatal development is detected. In some such embodiments, expression of one or more genes selected from the group consisting of neuropeptide Y receptor Y1 (NPY1R); leptin receptor (LEPR); growth hormone secretagogue receptor (GHSR); prostaglandin E receptor 3 (subtype EP3) (PTGER 3); hypocretin (orexin) receptor 2 (HCRTR2); galanin receptor 3 (GALR3); lactalbumin alpha (LALBA); glucagon (GCG); melanin-concentrating hormone receptor 1 (MCHR1); prostaglandin E receptor 3 (PTGER3); cholecytokinin A receptor (CCKAR); odorant binding protein 2B (OBP2B); transient receptor potential cation channel, subfamily V, member 1 (TRPV1); taste receptor, type 2, member 1 (TAS2R1); surfactant protein B (SFTPB); cystic fibrosis transmembrane conductance regulator (CFTR); fibroblast growth factors (FGF) 1, 2, 7, 10, 18; fibroblast growth receptor 2 (FGFR2); and combinations thereof is detected and/or identified.

In some embodiments, expression of one or more genes downregulated during neonatal development is detected. In some such embodiments, expression of one or more genes selected from the group consisting of carcinoembryonic antigen-related cell adhesion molecule 1 (biliary glycoprotein) (CEACAM1); V-raf murine sarcoma viral oncogene homolog B1 (BRAF); amino-terminal enhancer of split (AES); E1A binding protein p300 (EP300); Fas (TNF receptor superfamily member 6) (FAS); Fas (TNFRSF6)-associated via death domain (FADD); cyclin-dependent kinase inhibitor 2A (melanoma, p16, inhibits CDK4) (CDKN2A); glycogen synthase kinase 3 Beta (GSK3B); protein kinase, cAMP-dependent, regulatory, type 1, alpha (tissue specific extinguisher 1) (PRKAR1A); signal transducer and activator of transcription 5B (STAT 5B); aryl hydrocarbon receptor nuclear translocator (ARNT); insulin receptor (INSR); and combinations thereof is detected and/or identified.

In some embodiments, expression of genes from the aforementioned list and/or genes identified using methods of the invention is used together with expression of known genes involved in particular processes to determine a diagnosis.

In some embodiments, expression of genes known or discovered to be involved in a disease or condition (for example, neonatal development and particularly neonatal feeding characteristics) are also detected and used in a determination of the relevant diagnosis. In some embodiments, expression of one or more genes selected from the group consisting of (NKκB), I kappa B-alpha (IκB-α), toll-like receptor 4 (TLR4), platelet activating factor (PAF), platelet activating factor acetylhydrolase (PAF-AH), interleukin 8 (IL-8), epidermal growth factor (EGF), interleukin 10 (IL-10), endothelial 1 (ET-1), and combinations thereof are also detected and/or identified.

In some embodiments, expression of one or more of the following genes using methods of the invention are used to determine a diagnosis: AMP-activated protein kinase (AMPK), eukaryotic translation initiation factor 3 subunit D (EIF3D), adiponectin receptor 1 (ADIPOR1), leptin receptor overlapping transcript-like 1 (LEPROTL1), plexin A1 (PLXN1), olfactory receptor family 7 subfamily E member 156 pseudogene (OR7E156P), YY1 transcription factor (YY1), potassium invardly-rectifying channel subfamily J member 10 (KCNJ10), solute carrier family 6 (neurotransmitter transporter, creatine) member 8 (SLC6A8), integrin beta-1 (ITGB1), distal-less homeobox 2 (DLX2), SRY (sex determining region Y)-box 9 (SOX9), Kv channel interacting protein 3 calsenilin (KCNIP3), amyloid beta (A4) precursor-like protein 2 (APLP2), neurofibromin 2 (NF2), unc-5 homolog A (C. elegans) (UNC5A), wingless-type MMTV integration site family member 3 (WNT3), zinc finger and BTB domain containing 7A (ZBTB7A), inhibin beta A (INHBA), sonic hedgehog (SSH), teashirt zinc finger homeobox 3 (TSHZ3), BMI1 polycomb ring finger oncogene (BMI1), vasoactive intestinal peptide receptor 2 (VIPR2), insulin receptor (INSR), integrin beta 1 (ITGB1), and combinations thereof.

### Diagnosis

Determining a diagnosis of a neonate may involve making a determination with respect to the developmental progress of the neonate. Developmental progress may relate to such factors as the neonate's feeding capabilities, such as readiness to feed (readiness to transition from enteral feeding to oral feeding) and/or feeding tolerance (ability to establish and/or maintain full enteral feeding). Developmental progress may be assessed in relation to other factors such as ability to breathe independently and/or with a coordinated rhythm, etc.

Determining a diagnosis of a neonate can involve, among other things, determining that the neonate is susceptible for a condition or disease, that the neonate is developing the condition or disease, that the neonate has the condition or disease, that the neonate has a particular stage of the condition or disease, and/or that the neonate's condition is improving or recovering from a disease.

The condition or disease that may be determined may relate to problems of development, neurodevelopment, breathing, feeding, etc. For example, the disease may relate to problems in the digestive system, which may be underdeveloped in the neonate, and which relate to feeding. Such conditions or disease often affect prematurely born neonates. In some embodiments of the invention, the condition or disease that is determined is selected from the group consisting of necrotizing enterocolitis, respiratory distress syndrome, bronchopulmonary dysplasia, sepsis, and combinations thereof.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Identification of Appropriate Reference Genes for Normalization of Gene Expression Data

As mentioned previously, genes that appear to be associated with either a protective or harmful effect on neonatal feeding pathology are of particular interest to the present inventors. Expression of such genes will be confirmed using real time RT-PCR. Relative quantification of expression levels using real time RT-PCR requires choosing an appropriate reference gene whose expression levels can be used to normalize data.

In this Example, three reference genes: glyceraldehyde-3-phosphate dehydrogenase (GAPDH), tyrosine 3-monoxygenase/tryptophan 5-monooxygenase activation protein, zeta polypeptide (YWHAZ), and hypoxanthine phosphoribosyltransferase 1 (HPRT1) were selected for normalization of data from neonatal salivary samples. Reference genes were selected based upon microarray data from previous studies conducted in the inventors laboratory that revealed that each of these genes maintains a relative constant and consistent range of expression across newborns with different post-conceptual ages (PCAs).

### Examples 2-4

### Gene Expression Analyses on Neonatal Saliva Samples

Whole transcriptome microarrays are used in each of Examples 1-3. Although the analyses in the following Examples are initially focused on neonatal feeding and related complications, data generated from the Examples help build a library of banked neonatal transcriptomic information. Development of this library is also a long term goal of the experiments described below. Such a library may provide an invaluable resource for retrospective focused analyses of different neonatal complications and may contribute to our overall understanding of neonatal developmental genomic and network pathways.

### Example 2

### Gene Expression Analyses on Neonatal Saliva Samples and Identification of Genes Involved in Feeding

The experiments described in this Example illustrate that RNA can be successfully extracted and amplified from neonatal saliva samples and used in gene expression profiling experiments. Furthermore, experiments in this Example identified a limited list of genes whose expressions were differentially regulated in neonates who were feeding (at time of sample collection) compared those who were not. Among the list of differentially expressed genes are genes encoding digestive enzymes and neurodevelopmental genes. These results confirm that gene expression profiling of saliva samples can uncover physiologically relevant genes and suggest that biomarkers involved in particular processes, disease states, and/or conditions can be identified using such methods. Specific hypotheses relating to the involvement of particular genes or types of genes in such processes, disease states, and/or conditions may be tested using experimental paradigms similar to those used in this Example.

To date, 247 neonates ranging in gestational ages from about 24 6/7 weeks to 42 1/7 weeks have been enrolled in this study, and over 700 salivary samples have been obtained by suctioning from the neonate's oropharynx. Each sample comprised approximately 10 μL up to 200 μL of saliva.

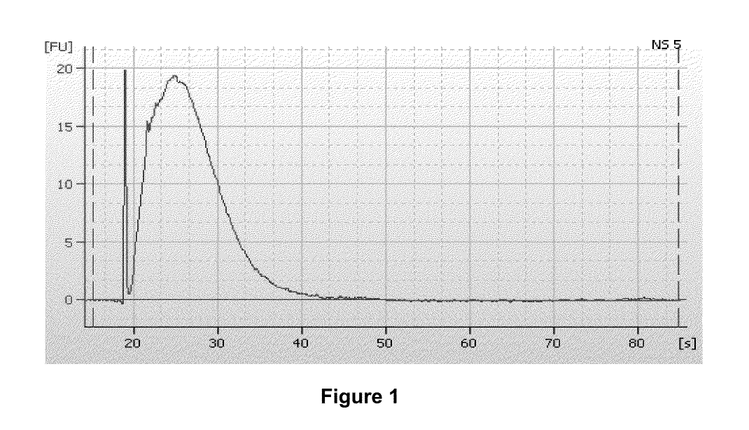

Total RNA was extracted from each sample and stored at −80° C. until further use. As depicted in FIG. 1, neonatal salivary RNA was successfully amplified in quantities more than sufficient for further experiments, demonstrating that extracted RNA was of high quality. FIG. 1 shows representative BioAnalyzer result of amplified total RNA from neonatal saliva sample. Following amplification, concentrations of starting RNA material ranged from about 600 ng/μL to about 3,200 ng/μL.

Five infants were selected for microarray analyses. These infants had a relatively benign neonatal course and did not have significant gastrointestinal sequelae. Their pertinent clinical information can be found in Table 1.

TABLE 1
clinical characteristics of subjects selected
for initial microarray analyses

| | | Gestational age | Birth weight |
|---|---|---|---|
| Subject | Gender | at birth (weeks) | (grams) |
| 1 | Male | 29 0/7 | 1389 |
| 2 | Female | 28 3/7 | 942 |
| 3 | Male | 28 3/7 | 1123 |
| 4 | Female | 32 0/7 | 1683 |
| 5 | Female | 32 0/7 | 1379 |

For each infant, five microarrays were run from salivary RNA obtained from the following time points: 1) shortly after birth and prior to enteral feeds, 2) at initiation of enteral feeds, 3) at full enteral nutrition, 4) at start of oral feeding, and 5) at full or majority oral feeding. For each sample, 5 ng of amplified and labeled RNA was hybridized onto an Affymetrix HG U133 Plus 2.0 whole genomic microarray. Hybridization rates for arrays ranged from about 7% to about 32%. Calculations were done in R version 2.8.1, a computer language program within Bioconductor version 2.3 (Gentleman et al. (2004), the entire contents of which are herein incorporated by reference) and lme4 (Bates et al., the entire contents of which are herein incorporated by reference). (For more information about R, see the website whose address is “http:” followed immediately by “//www.r-project.org/”.) Probe sets were summarized and arrays normalized using the rma( ) function in the Bioconductor affy package with default settings (Gautier et al. (2004), the entire contents of which are herein incorporated by reference). For each probe set, the significance of gestational age was determined by fitting two statistical models. The first model fit a random subject effect. The second model fit a linear age effect and a random subject effect. The two models were compared using the anova( ) function in R, using the likelihood ratio test. Significant p-values were then adjusted for a false discovery rate (FDR) using the Benjamini-Hochberg procedure (Benjamini and Hochberg (1995), the entire contents of which are herein incorporated by reference). Probe sets were identified as significantly differentially expressed for age when the FDR p-value was less than 0.05.

Of the 54,675 transcripts on the array, 9,286 showed significant expression changes over time (i.e., −a p-value less than 0.05). Key biomarkers of interest of this study, including EGF, IL8, TLR, and PAF were all detected in the saliva and found to significantly change over time. Related genes including the MO receptor and EGF receptor were also identified. These results confirmed that single genes could be analyzed using gene expression array technology.

In addition to single gene analysis, data were used to analyze genes on a global level and to shed light on possible interactions between gene products. Based on calculated T-scores, the significant gene list was divided into those showing a trend towards decreased expression over time (negative T score; n=3522), and those showing a trend in increased expression over time (positive T score; n=5764). Each respective gene list was then entered into Ingenuity Pathway Analysis® (IPA) for a formal, comprehensive analysis. Ingenuity® is an integrated commercially available database that allows researchers to search, explore, visualize, and analyze biological and chemical findings related to genes, proteins, and small molecules (e.g., drugs). IPA assesses how individual genes within a group relate to one another and calculates statistically over-represented systems within such a described list. Significant over-represented networks are group into one or more categories: Physiological System Development and Function, Molecular and Cellular Functions, Disease and Disorders, Toxicity Pathways, and Canonical Pathways. The top 5 up-regulated and down-regulated physiological development systems identified with IPA are depicted in Tables 2 and 3, respectively.

TABLE 2
Top 5 up-regulated physiological development systems

| | Approximate | # of |
|---|---|---|
| Physiological System | P-values | Genes |
| Behavior | from ~1.2 × 10⁻¹¹ | 170 |
| | to ~1.5 × 10⁻² | |
| Nervous System | from ~1.8 × 10⁻⁹ | 407 |
| Development and Function | to ~1.7 × 10⁻² | |
| Tissue Development | from ~4.6 × 10⁻⁷ | 226 |
| | to ~1.7 × 10⁻² | |
| Organ Development | from ~6.0 × 10⁻⁶ | 227 |
| | to ~1.6 × 10⁻² | |
| Digestive System | from ~1.2 × 10⁻⁵ | 49 |
| Development and Function | to ~1.3 × 10⁻² | |

TABLE 3
Top 5 down-regulated physiological development systems

| | Approximate | # of |
|---|---|---|
| Physiological System | P-values | Genes |
| Embryonic development | from ~7.2 × 10⁻¹⁴ | 128 |
| | to ~1.2 × 10⁻³ | |
| Connective Tissue | from ~4.8 × 10⁻⁸ | 147 |
| Development and Function | to ~2.3 × 10⁻³ | |
| Hematological System | from ~1.3 × 10⁻⁷ | 225 |
| Development and Function | to ~2.3 × 10⁻³ | |
| Hematopoiesis | from ~1.3 × 10⁻⁷ | 122 |
| | to ~9.2 × 10⁻⁴ | |
| Organismal Survival | from ~6.5 × 10⁻⁷ | 173 |
| | to ~2.1 × 10⁻³ | |

As can be seen from Tables 2 and 3, neonatal salivary transcriptomic analysis can indeed provide a window into the premature infant's gastrointestinal development and neurodevelopment as an infant learns to orally feed. Furthermore, it was unexpectedly discovered that transcriptomic analysis of neonatal saliva provides a picture of overall global development of a developing premature infant.

Among the over 9,000 genes that were differentially expressed during the course of infant development (that is, during the days after birth of the premature infant), genes with the most highly significant (p<0.001) expression differences were identified. These included both upregulated and downregulated genes and are discussed further below.

### Highly Significantly Upregulated Genes

A number of highly significantly upregulated genes are involved in development of the digestive system and/or in digestion. Several of these upregulated genes have functions relating to feeding. For example, neuropeptide Y receptor Y1 (NPY1R) was found to be upregulated over time. Neuropeptide Y is one of the most abundant neuropeptides in the mammalian system, with a diverse range of important physiologic functions, including food intake. Other upregulated genes include Leptin Receptor (LEPR), a receptor to an adipocyte-specific hormone that regulates adipose tissue mass through hypothalamic effects on satiety and energy; growth hormone secretagogue receptor (GHSR), which may play a role in energy homeostasis and regulation of body weight; and prostaglandin E receptor 3 (subtype EP3) (PTGER 3), which may have many biological functions involving digestion, the nervous system, kidney reabsorption, and uterine contraction activities.

Highly significantly upregulated genes involved in digestion also featured genes involved in feeding behavior, such as hypocretin (orexin) receptor 2 (HCRTR2), a G-protein coupled receptor involved in the regulation of feeding behavior. Orexins are believed to be primarily involved in stimulation of food intake, wakefulness, and energy expenditure. Galanin receptor 3 (GALR3), a neuropeptide that modulates a variety of physiologic processes including cognition, sensory/pain processing, hormone secretion, and feeding behavior, was also found to be upregulated. Lactalbumin alpha (LALBA) and glucagon (GCG) were also upregulated. Alpha lactalbumin is a principal protein of milk and forms the regulatory subunit of the lactose synthase heterodimer that enables production of lactose by transferring galactose moieties to glucose. Glucagon is a pancreatic hormone that counteracts the glucose-lowering action of insulin by stimulating glycogenolysis and gluconeogenesis.

Additionally, there were 407 gene transcripts involved in nervous system development whose up-regulation over time was highly significant. These gene transcripts were involved in a broad range of aspects of nervous system development, including development of neurons, nerves, the central nervous system, and nervous tissue; formation of oligodendrocytes and neuroglia; growth of neurites; and myelination. One particular nerve was highlighted among these genes: the trigeminal nerve (CN V). Genes that were upregulated over time included some that were involved in three specific functions or aspects associated with trigeminal nerve's: development of trigeminal ganglion nerves, quantity of trigeminal ganglion neurons, and survival of trigeminal ganglion neurons. The trigeminal nerve transmits somatosensory information (such as touch and pain) from the face and head and innervates muscles involved in chewing. Genes involved in olfactory system development (including development of olfactory bulb and of olfactory receptor neurons) were also upregulated in a highly significant manner.

Feeding associated genes that displayed highly significant upregulation over time included receptors involved in regulating food consumption. These genes included melanin-concentrating hormone receptor 1 (MCHR1), which is likely involved in neuronal regulation of food consumption; prostaglandin E receptor 3 (PTGER3), a receptor that has many biological functions including digestion, nervous system, kidney reabsorption, and uterine contraction activities; and cholecytokinin A receptor (CCKAR), a major physiologic mediator of pancreatic enzyme secretion and smooth muscle contraction of the gallbladder and stomach. In the central and peripheral nervous system, cholecytokinin A receptor regulates satiety and the release of beta-endorphin and dopamine.

Genes involved in sniffing were also found to be highly significantly upregulated and included odorant binding protein 2B (OBP2B); transient receptor potential cation channel, subfamily V, member 1 (TRPV1); and taste receptor, type 2, member 1 (TAS2R1). TRPV1 encodes a receptor for capsaicin, an ingredient that elicits a sensation of burning pain. The receptor conveys information about noxious stimuli to the central nervous system and is also activated by increases in temperature in the noxious range, which may indicate that it functions as a transducer of painful thermal stimuli in vivo. TAS2R1 encodes a member of a family of candidate taste receptors that belong to the G protein coupled receptor superfamily and that are specifically expressed by taste receptor cells of the tongue and palate epithelia.

Several genes involved in respiratory development were also highly significantly upregulated. These genes include surfactant protein B (SFTPB), an amphipathic surfactant protein essential for lung function and homeostasis after birth; cystic fibrosis transmembrane conductance regulator (CFTR), a chloride channel that controls regulation of other transport pathways; fibroblast growth factors (FGF) 1, 2, 7, 10, 18, which have broad mitogenic and cell survival activities and are involved in a variety of biological processes (including embryonic development, cell growth, morphogenesis, tissue repair, tumor growth, and invasion); and fibroblast growth receptor 2 (FGFR2), which has been implicated in diverse biological processes such as limb and nervous system development, wound healing, and tumor growth.

### Highly Significantly Downregulated Genes

A number of highly significantly downregulated genes are involved in embryonic development. One such gene is carcinoembryonic antigen-related cell adhesion molecule 1 (biliary glycoprotein) (CEACAM1), a cell-cell adhesion molecule detected on leukocytes, epithelia, and endothelia. CEACAM1 is involved in the arrangement of tissue three-dimensional structure, angiogenesis, apoptosis, tumor suppression, metastasis, and modulation of innate and adaptive immune responses. Another embryonic development gene identified as being downregulated is V-raf murine sarcoma viral oncogene homolog B1 (BRAF), which plays a role in regulating the MAP kinase/ERK signaling pathway, which affects cell division, differentiation, and secretion. Mutations in BRAF are associated with cardiofaciocutaneous syndrome. Other down-regulated genes included amino-terminal enhancer of split (AES), which is involved in neurogenesis during embryonic development; E1A binding protein p300 (EP300), which has been identified as a co-activator of HIF1A (hypoxia-inducible factor 1 alpha) and plays a role in stimulating hypoxia induced genes such as VEGF; Fas (TNF receptor superfamily member 6) (FAS), a receptor that contains a death domain, has been shown to play a central role in the physiological regulation of programmed cell death, and has been implicated in the pathogenesis of various malignancies and diseases of the immune system; and Fas (TNFRSF6)-associated via death domain (FADD), an adaptor molecule that interacts with various cell surface receptors and mediates cell apoptotic signals. FADD knockout studies in mice suggest the importance of FADD in early T cell development.

Another set of highly significantly downregulated genes are involved in organismal survival. One such gene is cyclin-dependent kinase inhibitor 2A (melanoma, p16, inhibits CDK4) (CDKN2A), a stabilizer of the tumor suppressor protein p53. CDKN2A is frequently mutated or deleted in a wide variety of tumors and is known to be an important tumor suppressor gene. Other downregulated genes include glycogen synthase kinase 3 Beta (GSK3B), a phosphorylating and inactivating glycogen synthase that is involved in energy metabolism, neuronal cell development, and body pattern formation; protein kinase, cAMP-dependent, regulatory, type 1, alpha (tissue specific extinguisher 1) (PRKAR1A), a tissue-specific extinguisher that down-regulates expression of seven liver genes in hepatoma-fibroblast hybrids; signal transducer and activator of transcription 5B (STAT5B), which mediates signal transduction triggered by various cell ligands (such as IL2, IL4, CSF1, and different growth hormones) and is involved in diverse processes (such as TCR signaling apoptosis, adult mammary gland development, and sexual dimorphism of liver gene expression); aryl hydrocarbon receptor nuclear translocator (ARNT), which is involved in induction of several enzymes that participate in xenobiotic metabolism and is identified as the beta subunit of a heterodimeric transcription factor (hypoxia-inducible factor 1; and insulin receptor (INSR), which together with its ligand insulin stimulates glucose uptake.

These experiments identified genes involved in neonatal development of premature infants, including genes involved in feeding. Furthermore, these results confirm that in addition to allowing analysis of a single gene or protein of interest, microarray technology also facilitates analysis of interactions between multiple related genes during normal postnatal development and/or in the presence of disease.

### Examples 3-4

### Profiling to Examine Readiness to Feed and Tolerance of Feeding

In these Examples, neonatal salivary genomic expression profiles are obtained and used to provide novel and informative data regarding development and physiological conditions related to feeding. Experiments described in these Examples are expected to identify certain genes and/or sets of genes as biomarkers that can be used to make certain determinations. These determinations may include, among other things, whether a neonate is ready to feed, a neonate's tolerance of feeds, and/or whether a neonate is at risk for developing, has developed, or is in a particular stage of a disease or condition.

### Target Population for Enrollment

Neonates born at or after 23 weeks and up to term gestation are targeted for enrollment. While the younger infants have an increased likelihood of developing feeding intolerance due to their prematurity at birth, infants born at a later gestational age will need to acquire the skills required for successful oral feeding prior to discharge to home. It is intended in these studies to capture neonates as they begin orally feeding. At the Floating Hospital for Children's NICU, where these studies are conducted, it is the general practice to introduce oral feeding at ≧33 weeks' gestation. In addition, a subset of newborns born at term gestation will have difficulty orally feeding. Those infants will also be a target of this study.

### Acquisition of Saliva and Selection of Neonates for Gene Expression Microarray Experiments

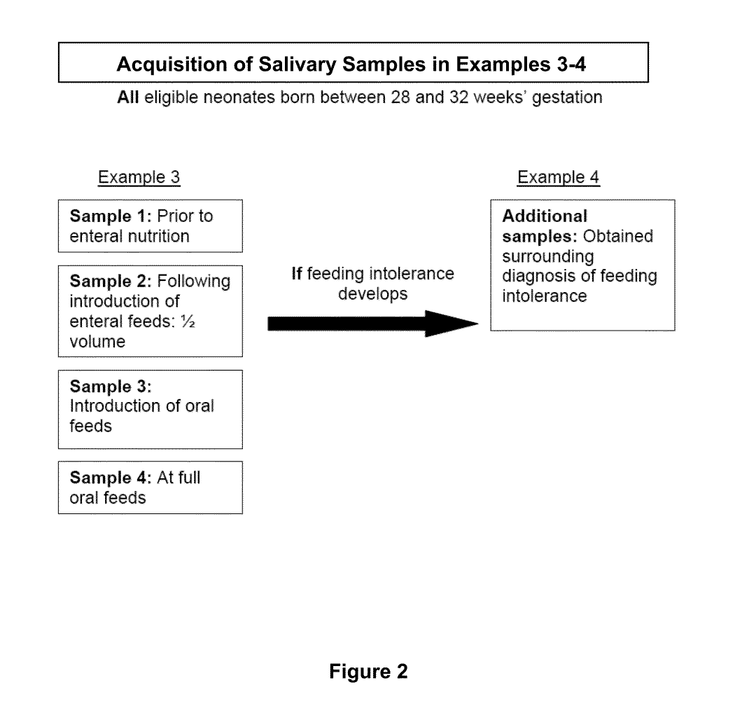

Saliva is obtained serially for all enrolled neonates throughout their hospitalizations. Because oral suctioning of neonates is part of routine neonatal care in the NICU, and the obtainment of saliva samples is expected to pose no threat to the neonates. A timeline for saliva acquisition for experiments described in these Examples is depicted in FIG. 2.

Samples are intentionally acquired repetitively in these studies for at least two reasons. First, as stated previously, neonates enrolled in these studies may develop other complications of prematurity. Collecting serial samples from the same neonate over time affords a possible way to control for such variations. Second, expression levels of genes of interest may fluctuate. While some genes (such as, for example, reference genes) may show little variation from day to day or week to week, other genes (such as, for example, neurodevelopmental genes and genes involved in inflammation) are often dynamically expressed. Sampling saliva from the same neonates serially may allow pinpointing specific genes involved in normal physiologic and/or in various pathological processes relevant to developmental pathways in the preterm neonate.

Salivary RNA from each neonate in these studies are obtained and stored. The decision to perform gene expression microarray experiments on particular neonates are made retrospectively (i.e., after clinical outcomes of the neonates are known). Neonates are selected for microarray expression analysis if complete sets of adequate salivary samples were obtained from them and if the neonates meet relevant clinical criteria for appropriate comparisons for each particular study. Salivary samples from neonates not selected for microarray expression analysis are appropriately processed and stored for possible subsequent use in developing a larger genomic expression data panel, a long range goal of this work.

### Statistical Analyses

It has been estimated that at least five gene expression microarray analyses may be needed to provide sufficient power for the intended analyses in these Examples. Therefore, for each Example, salivary samples from no fewer than 5 neonates are considered in each arm of the analysis.

Microarray data analyses are performed in R using the Affy and Multtest packages in Bioconductor (Gentleman R. C. et al. 2004). Array data are normalized using the quantile normalization method. ANOVAs are performed and p-values will be adjusted for multiple testing using the Benjamini-Hochberg false discovery rate approach (Benjamini and Hochberg (1995)). Candidate biomarkers are selected if their adjusted p-values are less than 0.05. Analyses of sets of genes in known pathways are also performed using Gene Set Enrichment Analysis (GSEA). (Romero and Tromp (2006), the entire contents of which are herein incorporated by reference in their entirety.) This analytical method can identify subtle but consistent gene expression changes in previously defined pathways. Once lists of genes with statistically significant expression differences are generated for each comparison, information (e.g., functional roles and expression patterns) about each gene in the list from publically available databases (e.g., OMIM, www.ncbi.nlm.nih.gov/sites/entrez?db=OMIM; PubMed, www.ncbi.nlm.nih.gov/sites/entrez; NetAffx, www.affymetrix.comianalysis/index.affx; UniGene, www.ncbi.nlm.nih.gov.sites/entrez?db=unigene; etc.), as well as commercially available databases, (e.g. Ingenuity), are manually reviewed to determine the potential role of each gene in development related to feeding and physiological readiness to feed.

### Example 3

### Identification of Genes that May be Used as Biomarkers of a Neonate's Readiness to Feed

In this Example, neonatal salivary genomic expression profiles are obtained and used to provide novel and informative data regarding a neonate's readiness to feed. Saliva samples are collected from enrolled neonates at particular timepoints: prior to the initiation of enteral feeding, following introduction of enteral feeds, and during the learning process of oral feeding. Expression profiles of developmental genes are chronicled in the developing preterm neonate by analyzing samples from such timepoints. Experiments described in this Example may identify mucosal, mesenchymal, and neurodevelopmental genes whose transcripts are expressed as neonates begin to orally feed. Such genes may be useful as biomarkers to determine a neonate's readiness to feed.

After parental consent and Health Insurance Portability and Accountability Act (HIPAA) authorization, each neonate and all corresponding salivary samples are assigned a code known only to the Principal Investigator and research assistant(s). Salivary samples are obtained at four time points of interest: 1) prior to the initiation of enteral feeds; 2) following the introduction of enteral feeds once a neonate reaches half volume of full feeds; 3) at the introduction of oral feeding; and 4) at full oral feeds. For each time point, the oropharynx of the neonate is gently suctioned to collect approximately 10 μL up to approximately 200 μL of saliva just prior to a feed to reduce the risk of contamination from formula or breast milk. Salivary samples are immediately stabilized with Qiagen™ RNAprotect Saliva Reagent. Salivary RNA extractions are subsequently performed with the commercially available Qiagen RNEasy® Protect Saliva kit. Extracted salivary RNA is stored at −80° C. until future analysis.

For microarray analysis, stored extracted salivary RNA is amplified, biotinylated, and fragmented with the Nugen™ Pico Amplification and Biotinylation and Fragmenting kits. Quality and quantity of amplified salivary samples is assessed with the Agilent™ BioAnalyzer 2100. Approximately 5 μg of amplified salivary mRNA is then hybridized onto the Affymetrix™ HGU133 Plus 2.0 array. Arrays are washed, stained, and scanned. Bioinformatic analyses is performed on the microarray data to identify genes whose expression levels differ among the time points of saliva collection in this study. Expression of genes that are identified as differentially expressed and that are believed to play a key role in the development of normal oral feeding patterns is quantified further by RT-PCR. Real-time RT-PCR is performed on remaining, stored, unamplified salivary samples by TaqMan™ amplification on an Applied Biosystem™ 7900 Sequence Detection System.

Gene expression levels of first, second, third, and fourth samples from each neonate in this study are compared using ANOVAs. The experiments in this Example may identify genes that are consistently changing between at least one pair of these groups of samples, and ultimately identify key mesenchymal genes necessary for the proper processing of enteral nutrition. Key neurodevelopmental genes necessary for successful oral feeding and gut motility are also expected to be identified in this study.

### Example 4

### Identification of Genes that May be Used as Biomarkers of Feeding Intolerance

In this Example, neonatal salivary genomic expression profiles are obtained and used to provide novel and informative data regarding the pathophysiology of feeding intolerance. Data from neonates who demonstrate feeding intolerance will be compared against data from those who do not. Without wishing to be bound by any particular theory, it is contemplated that longitudinal transcriptomic analyses of feeding-intolerant neonates will demonstrate upregulation of inflammatory (e.g., cytokines) and/or allergic (e.g., IgE) markers and/or disregulation of essential digestive enzymes. Such differentially or disregulated genes may potentially be used as biomarkers to differentiate between true pathology and more benign conditions. For example, it may be possible to distinguish, using such biomarkers, neonates suffering from a true formula allergy from neonates who may have an evolving pathological condition. Identification of genes involved in the pathophysiology of feeding intolerance may also allow prospective identification of some neonates who will subsequently develop a particular disease or condition.

In this Example, specific comparisons are also made between neonates who demonstrate feeding intolerance who are exclusively breastfed and those who are exclusively formula-fed. It is contemplated, without wishing to be bound by any particular theory, that salivary expression profiles between these cohorts of neonates should be different, and that comparative analysis allows identification of biomarkers within the breastfeeding group that may explain the presumed protective effect against the development of a particular disease or condition conferred upon premature breastfeeding neonates.

Samples are collected from neonates chosen for this study as described in the above “Target population for enrollment” section. In this Example, additional samples are collected from neonates who demonstrated feeding intolerance upon the introduction of enteral feeding. For the purposes of this Example, neonates are classified as feeding intolerant if the neonate has one or more of the following conditions: a) persistently heme positive stools without evidence of anal fissure or abrasions; b) abdominal distension warranting discontinuation of feeds or formula change; c) formula residuals representing 25% of initial feeds for at least 2 feeds within a 24 hour period; and d) inability to advance to or maintain full enteral feeds.

Statistical comparisons are made between gestational age-matched neonates who had no difficulty feeding and those who developed feeding intolerance as previously described. For comparative analysis, neonates in each group must have a complete set of adequate salivary RNA. For each group, two-way ANOVAs will be performed on salivary transcriptomic profiles on all available preceding time points. It is expected that by performing comparisons between groups of saliva collected over time, it is possible to identify discrepancies between neonates with feeding intolerance and those without for a particular time point. Additionally or alternatively, it may be possible to identify genetic markers of feeding intolerance whose expression change over time. Experiments and analyses described this Example may yield predictive markers that can be used to identify neonates at risk for developing feeding complications.

### Example 5

### Identification of Genes Involved in Hypothalamic Regulation that May be Used as Biomarkers of Oral Feeding Immaturity

In this Example, the expression profile of neuropeptide Y2 receptor, NPY2R, in relation to feeding status and post-conceptual age (PCA) was independently studied to determine its role as a biomarker in neonatal saliva to objectively predict successful oral feeding in the newborn. An important component of oral feeding success in the newborn is the developmental maturation of hypothalamic regulation of feeding behavior. Neuropeptide Y (NPY) and its family of five receptors are known to be associated with hypothalamic regulation of feeding behavior, metabolism, and energy homeostasis in both rodents and humans. For this Example, it was hypothesized that the physiological hyperhagia and exponential weight gain observed in healthy term newborns is associated with decreased expression of NPY2R. Therefore, persistence of NPY2R in salivary samples would suggest immature hypothalamic regulation, indicative of failed oral feeding trials. Results in this example confirmed that failure of a neonate to decrease NPY2R gene expression significantly correlated with an immature feeding pattern indicative of poor oral feeding skills. Neonates studied in this Example had a wide range of ages and clinical sequelae and this diverse patient population was essential in determining the applicability and accuracy of NPY2R as a diagnostic salivary biomarker. These results confirm that NPY2R is a highly novel biomarker in neonatal saliva that may be monitored noninvasively in order to objectively determine when an infant can be fed by mouth. Experiments and analysis described in this Example can be used for the development of an objective diagnostic tool that could be used prior to the introduction of oral feeds, particularly for those infants at risk for aspiration, hypoxia, and long-term feeding aversion.

### Results

### Demographics and Sample Characteristics

One hundred and sixteen salivary samples (10 to 50 μL) from 76 newborns with PCAs ranging from 26 4/7 to 41 4/7 weeks were collected. In this data set there were 63 preterm and 13 term infants and the pertinent clinical information for all subjects is shown in Table 4.

TABLE 4
Pertinent Clinical and Demographic Information

| Feeding | Number of | PCA | Weight | Summarized Medical Complications of |
|---|---|---|---|---|
| Stage | Subjects | (weeks) | (kg) | Subjects |
| 1 (NPO) | 17 | 25 3/7-36 1/7 | 0.73-2.136 | Respiratory distress syndrome (RDS), patent |
| | | | | ductus arteriosus (PDA), intrauterine growth |
| | | | | restriction (IUGR), bronchopulmonary |
| | | | | dysplasia (BPD), urinary tract infection |
| | | | | (UTI), neonatal abstinence syndrome (NAS), |
| | | | | pulmonary valvular stenosis, apnea, |
| | | | | hyperbilirubinemia, ABO incompatibility, |
| | | | | anemia, anal fissure, choanal atresia, |
| | | | | leukocytosis, metabolic acidosis, |
| | | | | undescended testicle, multiple gestation |
| 2 (PPG) | 21 | 28 2/7-41 3/7 | 0.78-3.845 | RDS, PDA, BPD, IUGR, apnea, anemia, |
| | | | | thrombocytopenia, coagulopathy, |
| | | | | hyperbilirubinemia, ABO incompatibility, |
| | | | | transient tachypnea, multiple gestation, |
| | | | | bacteremia, persistent pulmonary |
| | | | | hypertension (PPFTN), metabolic acidosis, |
| 3 (FPG) | 36 | 28 5/7-37 5/7 | 0.911-2.215 | RDS, IUGR, BPD, right grade I |
| | | | | intraventricular hemorrhage (IVH), |
| | | | | leukocytosis, acidosis, neutropenia, |
| | | | | peripheral pulmonary stenosis, transient |
| | | | | tachypnea, multiple gestation, narcotic |
| | | | | exposure, bacteremia, polydactyly |
| 4 (PPO) | 24 | 33 5/7-41 2/7 | 1.445-3.678 | RDS, NAS, IUGR, small for gestational age |
| | | | | (SGA), anemia, apnea, hypertension, |
| | | | | hemangioma, hyperbilirubinemia, multiple |
| | | | | gestation, twin-to-twin transfusion, |
| | | | | thrombocytopenia, anal fissure, membranous |
| | | | | choanal atresia, hypermagnesia |
| 5 (FPO) | 18 | 33 3/7-40 2/7 | 1.807-3.910 | Hyperbilirubinemia, ABO incompatibility, |
| | | | | RDS, BPD |

From the 76 subjects enrolled in this study, 31 had between two and five salivary samples analyzed, at either different PCAs and/or feeding statuses. The number of samples collected from subjects at each predefined feeding stage, as well as the percentage of infants expressing NPY2R, was as follows: Stage 1: no feeds (NPO) (n=17; PCA 25 3/7 to 36 1/7 weeks; 59% NPY2R expression); Stage 2: partial per gastric feeds (PPG) (n=21, PCA 28 2/7 to 41 3/7 weeks; 57% NPY2R expression); Stage 3: full per gastric feeds (FPG) (n=36, PCA 28 5/7 to 37 5/7 weeks; 67% NPY2R expression); Stage 4: partial oral feeds (PPO) (n=24, PCA 33 5/7 to 41 2/7 weeks; 50% NPY2R expression); Stage 5: full oral feeds (FPO) (n=18, PCA 33 3/7 to 40 2/7 weeks; 17% NPY2R expression).

Multiplex Reverse Transcription-Quantitative Polymerase Chain Reaction (qRT-PCR) Characteristics

Guidelines to the Minimum Information for Publication of Quantitative Real-Time PCR Experiments (MIQE) were followed (Bustin et al. (2009), the entire contents of which are herein incorporated by reference). All extracted total RNA samples were subjected to Multiplex qRT-PCR amplification for the gene NPY2R, along with three reference genes: GAPDH, YWHAZ, and HPRT1. Within the selected genes, one gene is known to be expressed at a relatively high level (YWHAZ), one at an average expression level (GAPDH), and one that demonstrates a low level of expression (HPRT1) at the limit of the detection level on the multiplex platform.

Prior to multiplex qRT-PCR, three samples were tested simultaneously on both uniplex and multiplex platforms for all genes to assess reaction efficiencies. No difference in reaction efficiency was observed in either of these assays. Since the quantification cycle (Cq) values for the three test samples run in both formats were within 1 cycle for all genes, it was concluded that running the samples on a multiplex platform would not impact the results. The reference gene YWHAZ had the highest level of expression in the tested samples (mean Cq: 27.7), followed by GAPDH (mean Cq: 30.3), and HPRT1 (mean Cq: 36.8). Mean delta Cq and standard deviation values between reference genes for all reactions were as follows: GAPDH-YWHAZ: 2.3+/−1.85; HPRT1-GAPDH 7.2+/−1.83; HPRT1-YWHAZ 9.5+/−1.93. These results demonstrate that reaction efficiencies across experiments are similar and reproducible. From among the 116 samples analyzed in this example, 95 had amplification of all three reference genes, while 21 revealed amplification of GAPDH, and YWHAZ only. And from among these 21 samples, six were positive for NPY2R expression and 15 had no detectable NPY2R in the sample.

### Assay Results

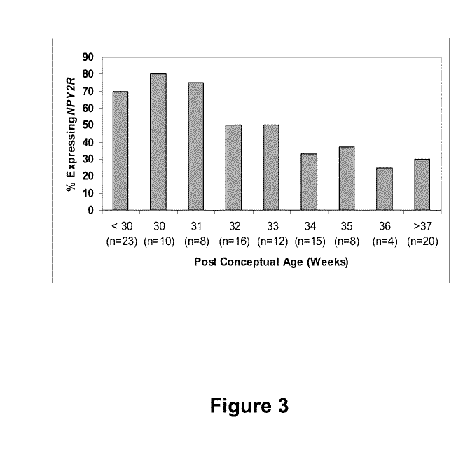

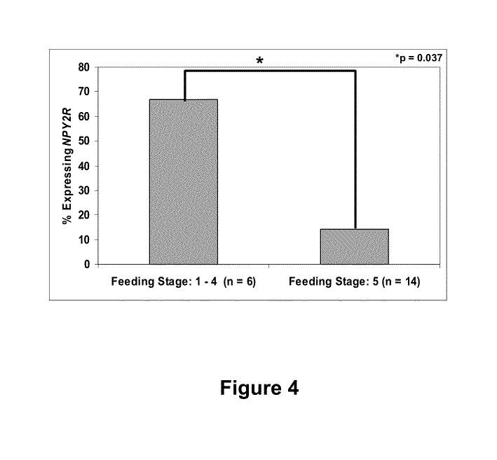

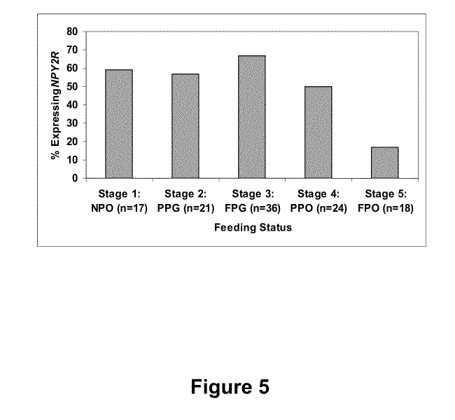

In this example, three separate statistical analysis were performed on the data. The first analysis considered all 116 data points; the second analysis considered only one sample per subject in order to ensure that multiple sampling from an individual(s) was not skewing the data (n=76); and the third analysis removed samples that did not have amplification of all three housekeeping genes in order to eliminate those samples that had a theoretical risk of a false negative result (n=95). NPY2R expression in neonatal saliva for all 116 samples had a 95% positive predictive value (CI: 85%-99%) of an immature feeding pattern with an inability to sustain full oral feeds (Stage 5). The negative predictive value of the assay was 27% (CI: 17%-41%) and the sensitivity was 59% (CI: 49%-69%) with 83% specificity (CI: 58%-96%). There was a statistically significant difference between NPY2R expression and PCA. Neonates that expressed NPY2R were younger than those infants that did not express the gene (p value<0.01) (FIG. 3). However, among term infants, there was a statistically significant difference between infants who could and could not orally feed (p value=0.037) (FIG. 4). Expression of NPY2R was associated with feeding status (p value=0.013) (FIG. 5), and as infants matured through the feeding stages, they were less likely to express the gene.

None of the two additional statistical analyses altered these primary results. Limiting the data set to include only one salivary sample per subject revealed a positive predictive value of 97% (CI: 85%-100%), with a negative predictive value of 38% (CI: 23%-55%). Sensitivity of the assay was 62% (CI: 50%-75%) with specificity of 93% (CI: 66%-100%). NPY2R expression remained statistically significantly associated with advancing PCA (Wilcoxon rank sum test p value<0.01) and feeding status (chi square p value=0.004). Further, eliminating the samples that did not have amplification of all three housekeeping genes in order to reduce the potential impact of false negative results had a positive predictive value of 95% (CI: 84%-99%), negative predictive value of 33% (CI: 20%-50%), with a sensitivity of 67% (CI: 55%-77%) and specificity of 81% (51%-95%). NPY2R expression was marginally statistically significantly associated with advancing PCA (Fisher's exact test p value=0.054). There remained a nonrandom association between NPY2R expression and feeding status (chi square p value=0.076).

### Materials and Methods

### Ethics Statement

This study was approved by the Tufts Medical Center Institutional Review Board. Written parental consent was obtained for all neonatal subjects enrolled.

### Demographics and Sample Characteristics

Salivary samples from premature and term neonates with a diverse range of clinical sequelae at various feeding stages during hospitalization were collected. The different stages were: Stage 1: no feeds (NPO); Stage 2: partial gastric feedings (PPG); Stage 3: full gastric feedings (FPG); Stage 4: partial oral feeds (PPO); Stage 5: full oral feeds (FPO). The feeding stage for the newborns at the time of collection of the salivary samples was solely determined by the caregivers and not influenced by study participation. The number of salivary samples collected from a newborn subject was dependent upon their clinical course. For most cases, the premature infants had more than one salivary sample obtained as they matured through the feeding process, while healthy term infants had only one salivary sample obtained at feeding stage 5, FPO. Also while the salivary samples were collected prospectively from all enrolled subjects, the correlation between salivary NPY2R gene expression and feeding status was made retrospectively once all samples were obtained and analyzed.

Salivary Collection and mRNA Extraction

All salivary samples were collected and processed according to the previously described standardized techniques that aim to simulate routine bedside care of the neonates (Dietz et al. (2011), the entire contents of which are herein incorporated by reference). Samples were stored at 4° C. for a minimum of 48 hours prior to total RNA extraction, which was performed with the RNA Protect Saliva Mini Kit (Qiagen™, Valencia, Calif. USA) per manufacturer's instructions. On column DNase digestion was performed on all samples to limit DNA contamination. Final elution volume was approximately 14 μL and the samples were stored at −80° C. until further analysis.

Multiplex qRT-PCR

All qRT-PCR experiments were performed on the Life Technologies 7900 instrument with the use of the Path-ID™ Multiplex One-Step RT-PCR Kit (Life Technologies, Carlsbad, Calif. USA). Standard stock sequences of reference genes were provided by Life Technologies and were VIC labeled, primer limited as follows: GAPDH-VIC (Hs03929097), HPRT1-VIC (Hs01003267), and YWHAZ-VIC (Hs03044281). Gene sequences for NPY2R were custom made with the use of Primer Express Software v 1 to ensure optimal G-C content and melting temperatures. The custom sequence for NPY2R (Sequence accession number: NM_{—}000910.2) was as follows: Forward Primer: GGC TTT CCT CTC GGC CTT C; Reverse Primer TGT CAC GGA CAC CTC AGA GTG; Probe 6FAM-CTG TGA GCA GCG GTT GGA TGC CAT-TAMRA. The amplicon is located at base pairs 1496 to 1563 of the gene and supposedly contains no SNPs. There are only two known exons for the NPY2R gene and the entire amplicon used in this study was contained within one exon.

For each salivary sample, NPY2R was run in triplicate, multiplexed one time each with the three reference genes. Negative controls with nuclease-free water were performed on each plate. The thermal cycle profile for all reactions was as follows: 48° C. for 10 minutes, 95° C. for 10 minutes, followed by 40 cycles of PCR with a 15 second denaturing cycle at 95° C., followed by 45 seconds of annealing and extension at 60° C. The total volume for each reaction was 25 μL, including 2.5 μL of template in each well.

For the purposes of this study, only the expression of NPY2R in neonatal saliva was considered. The gene was considered expressed if it amplified along with ≧2 of the reference genes. Similarly, NPY2R was considered not expressed if it did not amplify in the presence of ≧2 of the reference genes. Lacking any reference values for salivary NPY2R, it is difficult to provide a clinical interpretation based upon normalized relative quantitative values. Therefore, the data was analyzed in the context of expression of the gene in order to provide the most accurate and biologically relevant assessment. The binary nature of the assay makes it well suited for the development of a rapid diagnostic assay. Finally, average Cq for each reference gene was calculated, along with mean delta Cq and standard deviation values between housekeeping genes to assess efficiencies and variability between reactions.

### Statistical Analyses

Statistical analyses comprised of the Fisher's exact test to determine the relationship of NPY2R expression and gestational age, and the Wilcoxon rank sum test to compare gestational age in the groups of infants that did or did not express NPY2R (FIG. 3). Chi-square test was used to assess the association between NPY2R gene expression and feeding status in Stages 1-5 (FIG. 5). NPY2R expression at term gestation 37 weeks' gestation) was further examined with the use of a Fisher's exact test to compare expression of the gene in saliva between infants who could and could not successfully feed (FIG. 4). Sensitivity, specificity, negative and positive predictive values, along with each respective confidence interval, of the assay were then calculated. Additional statistical analyses were performed to: limit the analysis to those samples that had amplification of all three reference genes; and to ensure that repeat measures from the same individuals in this study did not skew the data. In the latter analysis, only one sample per infant was used, and this sample was determined by a random computer generated number to reduce the risk of bias.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### REFERENCES

1. Adams et al., “Complementary DNA sequencing: expressed sequence tags and human genome project.” Science. 1991, 252: 1651-1656
2. Aikens et al., “Solid-state imagers for microscopy.” Meth. Cell Biol. 1989, 29: 291-313
3. Alberts et al., “Molecular Biology of the Cell.” 1994 (3rd Ed.), Garland Publishing, Inc.: New York, N.Y.
4. Altschul et al., “Basic local alignment search tool.” J. Mol. Biol. 1990, 215(3): 403-410
5. Altschul et al., Methods in Enzymology
6. Altschul et al., Nucleic Acids Res. 1997, 25:3389-3402
7. Anderson (Ed.), “Nucleic Acid Hybridization.” 1999, Springer Verlag: New York, N.Y.
8. Anfinsen et al., “Studies on the gross structure, cross-linkages, and terminal sequences in ribonuclease.” J. Biol. Chem. 1954, 207: 201-210
9. Ausubel (Ed.), “Short Protocols in Molecular Biology.” 2002, 5th Ed., John Wiley & Sons;
10. Barringer et al., “Blunt-end and single-strand ligations by Escherichia coli ligase: influence on an in vitro amplification scheme.” Gene. 1990, 89: 117-122
11. Bassett et al., “Gene expression informatics—it's all in your mine.” Nat. Genet. 1999, 21: 51-55
12. Bates et al. “Ime4: Linear mixed-effects models using S4 classes. R package version 0.999375-28.” lme4.r-forge.r-project.org.
13. Baxevanis et al., “Bioinformatics: A Practical Guide to the Analysis of Genes and Proteins.” Wiley, 1998
14. Bayer et al., “The use of the avidin-biotin complex as a tool in molecular biology.” Methods of Biochem. Analysis. 1980, 26: 1-45
15. Becker-Andre and Hahlbrock, “Absolute mRNA quantification using the polymerase chain reaction (PCR). A novel approach by a PCR aided transcript titration assay (PATTY).” Nucl. Acids Res. 1989, 17: 9437-9346
16. Blackburn et al. “Ribonuclease inhibitor from human placenta. Purification and properties.” J. Biol. Chem. 1977, 252: 5904-5910
17. Benjamini and Hochberg. “Controlling the false discovery rate: a practical and powerful approach to multiple testing.” J R Stat Soc (Ser A) 1995 B75:290-300
18. Bravo et al., “A versatile negative-staining ribonuclease zymogram.” Anal. Biochem. 1994, 219: 82-86
19. Brazma and Vilo, “Gene expression data analysis.” FEBS Lett. 2000, 480: 17-24
20. Brison et al., “General method for cloning amplified DNA by differential screening with genomic probes.” Mol. Cell. Biol. 1982, 2: 578-587
21. Brigati et al., “Detection of viral genomes in cultured cells and paraffin-embedded tissue sections using biotin-labeled hybridization probes.” Virol. 1983, 126: 32-50
22. Britten et al., “Analysis of repeating DNA sequences by reassociation.” Methods in Enzymol., 1974, 29: 363-418
23. Broker et al., “Electron microscopic visualization of tRNA genes with ferritin-avidin: biotin labels.” Nucl. Acids Res. 1978, 5: 363-384
24. Burg et al., “Single molecule detection of RNA reporter probes by amplification with Q beta replicase.”Mol. Cell. Probes. 1996, 10: 257-271
25. Bustin S T, Benes B, Garson J A, Hellemans J, Huggett J et al. “The MIQE guidelines: Minimum information of publication of quantitative real-time PCR experiments.” Clin Chem 2009, 55:611-622.
26. Butler A A, Cone R D “Knockout models resulting in the development of obesity.” Trends Genet 2001, 17:S50-54.
27. Butte, “The use and analysis of microarray data.” Nat. Rev. Drug Discov. 2002, 1: 951-960
28. Chen et al., “Profiling Expression Patterns and Isolating Differentially Expressed Genes by cDNA Microarray System with Colorimetry Detection.” Genomics, 1998, 51: 313-324
29. Chen et al., “4,4-Difluoro-4-bora-3a,4a-diaza-s-indacene (BODIPY) Dyes Modified for Extended Conjugation and Restricted Bond Rotations.” J. Org. Chem. 2000, 65: 2900-2906
30. Chen et al., “Probing the cathepsin D using a BODIPY FL-pepstatin A: applications in fluorescence polarization and microscopy.” J. Biochem. Biophys. Methods, 2000, 42: 137-151
31. Cheung et al., “Making and reading microarrays.” Nature Genet. 1999, 21: 15-19
32. Chomczynski et al., “Single-step method of RNA isolation by acid guanidinium thiocyanate-phenol-chloroform extraction.” Anal. Biochem. 1987, 162: 156-159
33. Chiang et al., “Visualizing associations between genome sequences and gene expression data using genome-mean expression profiles.” Bioinformatics, 2001, 17: S49-S55
34. Chirgwin et al., Biochem. 1979, 18: 5294-529
35. Cloonan et al., “Transcriptome content and dynamics at single-nucleotide resolution.” Genome Biol. 2008, 9: 234.1-234.4
36. Connoly and P. Rider, “Chemical synthesis of oligonucleotides containing a free sulphydryl group and subsequent attachment of thiol specific probes.” Nucl. Acids. Res. 1985, 13: 4485-4502
37. Dewey, “From microarrays to networks: mining expression time series.” Drug Discov. Today, 2002, 7: S170-S175
38. Dietz J A, Johnson K J, Wick H C, Bianchi D W, Maron J L “Optimal techniques for mRNA extraction from neonatal salivary supernatant.” Neonatology 2011, 101:55-60.
39. Divane et al., “Rapid prenatal diagnosis of aneuploidy from uncultured amniotic fluid cells using 5 colour fluorescence in situ hybridization.” Prenat. Diagn. 1994, 14: 1061-1069
40. Duggan et al., “Expression profiling using cDNA microarrays.” Nat. Genet. 1999, 21: 10-14
41. Ekins and Chu, “Microarrays: their origins and applications.” Trends in Biotech. 1999, 17: 217-218
42. Gautier et al., “Affy-analysis of Affymetrix GeneChip data at the probe level.” Bioinformatics. 2004 20:307-315.
43. Gentleman et al. “Bioconductor: open software development for computational biology and bioinformatics.” Genome Biol. 2004 5: R(80).
44. Gilliland et al., “Analysis of cytokine mRNA and DNA: detection and quantitation by competitive polymerase chain reaction.” Proc. Natl. Acad. Sci. 1990, 87: 2725-2729
45. Glisin et al., “Ribonucleic acid isolated by cesium chloride centrifugation.” Biochem. 1974, 13: 2633-2637
46. Greijer et al., “Quantitative competitive NASBA for measuring mRNA expression levels of the immediate early 1, late pp 67, and immune evasion genes US3, US6, and US11 in cells infected with human cytomegalovirus.” J. Virol. Methods. 2001, 96: 133-147
47. Greiner-Stoeffele et al., “A General Ribonuclease Assay Using Methylene Blue.” Anal. Biochem. 1996, 240: 24-28
48. Grunert et al., “Trp59 to Tyr substitution enhances the catalytic activity of RNase T1 and of the Tyr to Trp variants in positions 24, 42 and 45.” Protein Eng. 1993, 6: 739-744
49. Guatelli et al., “Isothermal, in vitro amplification of nucleic acids by a multienzyme reaction modeled after retroviral replication.” Proc. Natl. Acad. Sci. 1990, 87: 1874-1878
50. Hamaguchi et al., “Aptamer Beacons for the Direct Detection of Proteins.” Anal. Biochem. 2001, 294: 126-131
51. Haugland, “Molecular Probes: Handbook of Fluorescent Probes and Research Chemicals 1992-1994”, 5th Ed., 1994, Molecular Probes, Inc.
52. Hayden, “Multiplex-ready PCR: a new method for multiplexed SSR and SNP genotyping.” BMC Biogenomics, 2008, 9:80
53. Hess et al., “Microarrays: Handling the deluge of data and extracting reliable information.” Trends Biotechnol. 2001, 19: 463-468
54. Hiraoka et al., “The Use of a Charge-Coupled Device for Quantitative Optical Microscopy of Biological Structures.” Science, 1987, 238: 36-41
55. Hopman et al., “Mercurated nucleic acid probes, a new principle for non-radioactive in situ hybridization.” Exp. Cell Res. 1987, 169: 357-368
56. Huang X-F, Yu U, Li Y, Tim S, Deng C, Wang Q “Ventromedial hypothalamic NPY Y2 receptor in the maintenance of body weight in diet-induced obesity in mice.” Neurochem Res 2008, 33:1881-1888.
57. Jalal et al., “Prenatal detection of aneuploidy by directly labeled multicolored probes and interphase fluorescence in situ hybridization.” Mayo Clin. Proc. 1998, 73: 132-137
58. Kerr et al., “Analysis of Variance for Gene Expression Microarray Data.” J. Comput. Biol. 2000, 7: 819-837
59. Marcotte and Date, “Exploiting big biology: integrating large-scale biological data for function inference.” Brief Bioinform. 2001, 2: 363-374
60. Ikehara et al., “Inquiries into the structure-function relationship of ribonuclease T1 using chemically synthesized coding sequences.” Proc. Natl. Acad. Sci. USA, 1986, 83: 4695-4699
61. Innis (Ed.), “PCR Protocols: A Guide to Methods and Applications.” Academic Press: New York, 1990
62. Innis (Ed.), “PCR Strategies.” Academic Press: New York, 1995
63. Joos et al., “Mapping and chromosome analysis: the potential of fluorescence in situ hybridization.” J. Biotechnol. 1994, 35: 135-153
64. Kaboev et al., “PCR hot start using primers with the structure of molecular beacons (hairpin-like structure).” Nucl. Acids Res. 2000, 28: E94
65. Kievits et al., “NASBA™ isothermal enzymatic in vitro nucleic acid amplification optimized for the diagnosis of HIV-1 infection.” J. Virol. Methods, 1991, 35: 273-286
66. Kimmel and Berger, “Preparation of cDNA and the generation of cDNA libraries: Overview.” Methods in Enzymol. 1987, 152: 307-316
67. Korn et al., “Analysis of the RNase T1 Mediated Cleavage of an Immobilized Gapped Heteroduplex via Fluorescence Correlation Spectroscopy.” Biol. Chem. 2000, 381: 259-263
68. Kricka, “Stains, labels and detection strategies for nucleic acids assays.” Ann. Clin. Biochem. 2002, 39: 114-129
69. Kwoh et al., “Transcription-based amplification system and detection of amplified human immunodeficiency virus type 1 with a bead-based sandwich hybridization format.” Proc. Natl. Acad. Sci. 1989, 86: 1173-1177
70. Landegren et al., “A ligase-mediated gene detection technique.” Science, 1988, 241: 1077-1080
71. Landegent et al., “2-acetylaminofluorine-modified probes for the hybridocytochemical detection of specific nucleic acid sequences.” Exp. Cell Res. 1984, 15: 61-72
72. Langer et al., “Enzymatic synthesis of biotin-labeled polynucleotides: novel nucleic acid affinity probes.” Proc. Natl. Acad. Sci., 1981, 78: 6633-6637
73. Liang and Pardee, “Differential display of eukaryotic messenger RNA by means of the polymerase chain reaction.” Science, 1992, 257: 967-971
74. Lin S, Boey D, Herzog H “NPY and Y receptors: lessons from transgenic and knockout models.” Neuropeptides 2004, 38:189-200.
75. Livak et al., “Oligonucleotides with Fluorescent Dyes at Opposite Ends Provide a Quenched Probe System Useful for Detecting PCR Product and Nucleic Acid Hybridization.” PCR Methods Appl. 1995, 4: 357-362
76. Lockhart and Winzeler, “Genomics, gene expression and DNA arrays.” Nature, 2000, 405: 827 836
77. Mansfield et al., “Nucleic acid detection using non-radioactive labelling methods.” Mol. Cell. Probes. 1995, 9: 145-156
78. Marshall and Hodgson, “DNA chips: an array of possibilities.” Nature Biotech. 1998, 16: 27-31
79. McClelland et al., “Arbitrary primed PCR fingerprinting of RNA applied to mapping differentially expressed genes.” EXS, 1993, 67: 103-115
80. Misener et al., (eds.), “Bioinformatics Methods and Protocols (Methods in Molecular Biology, Vol. 132)” Humana Press, 1999
81. Naveilhan P, Hassain H, Canals J M, Ekstrand A J, Larefalk A, et al. “Normal feeding behavior, body weight and leptin response require the neuropeptide Y Y2 receptor.” Nat Med 1999, 5:1188-93.
82. Neves et al., “Novel method for covalent fluorescent labeling of plasmid DNA that maintains structural integrity of the plasmid.” Bioconjugate Chem. 2000, 11: 51-55
83. Poddar and Le, “Bordetella pertussis detection by spectrofluorometry using polymerase chain reaction (PCR) and a molecular beacon probe.” Mol. Cell. Probes, 2001, 15: 161-167
84. Quaas et al., “Indicator plates for rapid detection of ribonuclease T1 secreting Escherichia coli clones.” Nucl. Acids Res. 1989, 17: 3318
85. R Development Core Team. “R: A language and environment for statistical computing. R Foundation for Statistical Computing.” 2008 Vienna, Austria. ISBN 3-900051-07-0, URL http://www.R-project.org.
86. Ramsay, “DNA chips: state-of-the art.” Nature Biotech. 1998, 16: 40-4
87. Richardson et al., “Biotin and fluorescent labeling of RNA using T4 RNA ligase.” Nucl. Acids Res. 1983, 11: 6167-6184
88. Sambrook et al., “Molecular Cloning: A Laboratory Manual”, 1989, 2nd Ed., Cold Spring Harbour Laboratory Press: New York)
89. Schena et al., “Quantitative monitoring of gene expression patterns with a complementary DNA microarray.” Science, 1995, 270: 467-470
90. Schena et al., “Parallel human genome analysis: microarray-based expression monitoring of 1000 genes.” Proc. Natl. Acad. Sci. 1996, 93: 10614-10619
91. Sebestyen et al., “DNA vector chemistry: the covalent attachment of signal peptides to plasmid DNA.” Nat. Biotechnol. 1998, 16: 568-576
92. Shi Y-C, Lin S, Wong I P L, Baldock P A, Aljanova A et al. “NPY neuron-specific Y2 receptors regulate adipose tissue and trabecular bone but not cortical bone in homeostasis in mice.” PLoS One 2010, 5:e11361.
93. Smith et al., “Detection of Mycobacterium tuberculosis directly from sputum by using a prototype automated Q-beta replicase assay.” J. Clin. Microbiol. 1997, 35(6): 1477-1491
94. Smith et al., “The synthesis of oligonucleotides containing an aliphatic amino group at the 5′ terminus: synthesis of fluorescent DNA primers for use in DNA sequence analysis.” Nucl. Acids Res. 1985, 13: 2399-2412
95. Sokol et al., “Real time detection of DNA•RNA hybridization in living cells.” Proc. Natl. Acad. Sci., 1998, 95: 11538-11543
96. Stern and Newton, “Isolation of plant mitochondrial RNA.” Methods in Enzymol. 1986, 118: 488
97. Su et al., “High-throughput RT-PCR analysis of multiple transcripts using a microplate RNA isolation procedure.” BioTechniques, 1997, 22: 1107-1113
98. Tchen et al., “Chemically modified nucleic acids as immunodetectable probes in hybridization experiments.” Proc. Natl. Acad. Sci. 1984, 81: 3466-3470
99. Tamames et al., “Bioinformatics methods for the analysis of expression arrays: data clustering and information extraction.” J. Biotechnol. 2002, 98: 269-283
100. Tarazona et al., “Differential expression in RNA-seq: a matter of depth.” Genome Res. 2011, 21: 2213-2223
101. Temsamani and Agrawal, “Enzymatic labeling of nucleic acids.” Mol. Biotechnol. 1996, 5: 223-232
102. Uddman R, Möller S, Nilsson T, Nyström S, Ekstrand J et al. “Neuropeptide Y Y₁ and neuropeptide Y Y₂ receptors in human cardiovascular tissues.” Peptides 2002, 23:927-934.
103. van Gijlswijk et al., “Universal Linkage System: versatile nucleic acid labeling technique.” Expert Rev. Mol. Diagn. 2001, 1: 81-91
104. Velculescu et al., “Serial analysis of gene expression.” Science, 1995, 270: 484-487
105. Wang et al., “Quantitation of mRNA by the polymerase chain reaction.” Proc. Natl. Acad. Sci. 1989, 86: 9717-9721
106. Wang L, Gourcerol G, Yuan P-Q, Wu S, Million M et al. “Peripheral peptide YY inhibits propulsive colonic motor function through Y2 receptor in conscious mice.” Am J Physiol Gastrointest Liver Physiolog 2010, 298:G45-G56.
107. Watson et al., “Technology for microarray analysis of gene expression.” Curr. Opin. Biotechnol. 1998, 9: 609-614
108. Welsh et al., “Arbitrarily primed PCR fingerprinting of RNA.” Nucl. Acids Res. 1992, 20: 4965-497
109. Wiegant et al., “ULS: a versatile method of labeling nucleic acids for FISH based on a monofunctional reaction of cisplatin derivatives with guanine moieties.” Cytogenet. Cell. Genet. 1999, 87: 47-52
110. Wilfinger et al., “Effect of pH and ionic strength on the spectrophotometric assessment of nucleic acid purity.” Biotechniques. 1997, 22: 474-481
111. Weinstein et al., “The bioinformatics of microarray gene expression profiling.” Cytometry. 2002, 47: 46-49
112. Xiang et al., “Microarray expression profiling: analysis and applications.” Curr. Opin. Drug Discov. Devel. 2003, 6: 384-395
113. Yamamoto et al., “Molecular beacon aptamer fluoresces in the presence of Tat protein of HIV-1.” Genes Cells. 2000, 5: 389-396
114. Yoo S B, Ryu V, Park E Y, Kim B-T, Kang D W et al. “The arcuate NPY, POMC, and CART expressions responding to food deprivation are exaggerated in young female rats that experienced neonatal maternal separation.” Neuropeptides 2011, 45:343-349.
115. Zhang et al., “Gene expression profiles in normal and cancer cells.” Science. 1997, 276: 1268-1272

### OTHER EMBODIMENTS

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method for detecting or identifying genes involved in a condition or disease affecting neonates comprising steps of:
providing a test sample of saliva RNA obtained from a neonate suffering from or diagnosed with a condition, wherein
the test sample has a volume of about 5 μL to about 50 μL;
subjecting the test sample of saliva RNA to an analysis, wherein the analysis comprises:
hybridizing the RNA to one or more oligonucleotide probes, such that one or more genes that are differentially regulated in the test sample as compared to a control sample is/are identified, wherein the control sample comprises saliva RNA obtained from a neonate that is not suffering from or diagnosed with the condition; and
determining that the one or more differentially regulated genes are involved in the condition or disease.

2. A method for detecting or identifying genes involved in neonatal development comprising steps of:
providing a test sample of saliva RNA obtained from a neonate, wherein
the test sample comprises a volume of about 5 μL to about 50 μL; subjecting the test sample of saliva RNA to an analysis, wherein the analysis comprises:
hybridizing the RNA to one or more oligonucleotide probes, such that one or more genes that are differentially regulated in the test sample as compared to a control sample is/are detected or identified, wherein the control sample comprises saliva RNA obtained from a neonate at a developmental stage different than the neonate from which the test sample of saliva RNA sample was obtained; and
determining that the one or more differentially regulated genes are involved in neonatal development.

3. A method for determining a diagnosis of a neonate comprising steps of:
providing a test sample of saliva RNA obtained from the neonate, wherein
the test sample comprises a volume of about 5 μL to about 50 μL; subjecting the test sample of saliva RNA to an analysis, wherein the analysis comprises:
hybridizing the RNA to one or more oligonucleotide probes, such that expression of at least one gene identified using the method of claim 1 is identified; and
determining, based on the detected expression of the at least one gene a diagnosis of the neonate.

4. A method for determining a diagnosis of a neonate comprising steps of:
providing a test sample of saliva RNA obtained from the neonate, wherein
the test sample comprises a volume of about 5 μL to about 50 μL; subjecting the test sample of saliva RNA to an analysis, wherein the analysis comprises:
hybridizing the RNA to one or more oligonucleotide probes, such that expression of at least one gene identified using the method of claim 2 is identified; and
determining, based on the detected expression of the at least one gene a diagnosis of the neonate.

5. A method for determining feeding capability of a neonate comprising steps of:
providing a test sample of saliva RNA obtained from a neonate; and
measuring expression of an NPY2R gene in the test sample, wherein
an elevated level of NPY2R gene expression in the test sample relative to a control indicates decreased feeding capability.

6. The method of claim 5, wherein the test sample comprises a volume of about 5 μL to about 50 μL.

7. The method of claim 5, wherein the feeding capability is selected from the group consisting of readiness to feed, feeding tolerance, and combinations thereof.

8. The method of claim 5, wherein the neonate is a premature neonate.

9. The method of claim 5, wherein the neonate is a term neonate.

10. A method for determining feeding capability of a neonate comprising steps of:
providing a test sample of saliva RNA obtained from a neonate; and
measuring expression of an NPY2R gene in the test sample, wherein
a decreased level of NPY2R gene expression in the test sample relative to a control indicates increased feeding capability.

11. The method of claim 10, wherein the test sample comprises a volume of about 5 μL to about 50 μL.

12. The method of claim 10, wherein the feeding capability is selected from the group consisting of readiness to feed, feeding tolerance, and combinations thereof.

13. The method of claim 10, wherein the neonate is a premature neonate.

14. The method of claim 10, wherein the neonate is a term neonate.

15. The method of claim 5, wherein the control is a sample of saliva RNA obtained from a neonate having a normal feeding capability.

16. The method of claim 10, wherein the control is a sample of saliva RNA obtained from a neonate having a normal feeding capability.
